SEQUENCE LISTING (14 sequences)

SEQ ID NO: 1 - DNA, 20 bp, Artificial Sequence
  Synthetic oligonucleotide
tcgtcattccattacctacc

SEQ ID NO: 2 - DNA, 24 bp, Artificial Sequence
  Synthetic oligonucleotide
tactgatcgataatgccagacgaa

SEQ ID NO: 3 - DNA, 24 bp, Artificial Sequence
  Synthetic oligonucleotide
aacgagctgtatgtcgtgagaatc

SEQ ID NO: 4 - DNA, 27 bp, Artificial Sequence
  Synthetic oligonucleotide
atggatcatcaagctctaagaaagaac

SEQ ID NO: 5 - DNA, 19 bp, Artificial Sequence
  Synthetic oligonucleotide
cgatcaggaaatcaaccag

SEQ ID NO: 6 - DNA, 20 bp, Artificial Sequence
  Synthetic oligonucleotide
ataggcttgaagcagtgcat

SEQ ID NO: 7 - DNA, 19 bp, Artificial Sequence
  Synthetic oligonucleotide
aggtaaccgaaaacaagcc

SEQ ID NO: 8 - protein, 224 aa, Pyrococcus furiosus
MKIGGIDEAGRGPAIGPLVVATVVVDEKNIEKLRNIGVKDSKQLTPHERKNLFSQITSIADDYKIVIVSPEEIDNRSGTMNELEVEKFALALNSLQIKPALIYADAADVDANRFASLIERRLNYKAKIIAEHKADAKYPVVSAASILAKVVRDEEIEKLKKQYGDFGSGYPSDPKTKKWLEEYYKKHNSFPPIVRRTWETVRKIEESIKAKKSQLTLDKFFKKP

SEQ ID NO: 9 - protein, 16 aa, Artificial Sequence
  Synthetic polypeptide
GIDEAGRGPAIGPLVV

SEQ ID NO: 10 - protein, 12 aa, Artificial Sequence
  Synthetic polypeptide
LRNIGVKDSKQL

SEQ ID NO: 11 - protein, 19 aa, Artificial Sequence
  Synthetic polypeptide
HKADAKYPVVSAASILAKV

SEQ ID NO: 12 - protein, 16 aa, Artificial Sequence
  Synthetic polypeptide
KLKKQYGDFGSGYPSD

SEQ ID NO: 13 - DNA, 1950 bp, Salmonella enterica
aacagtgctcgtttacgacctgaattactgattctggtactaatggtgatgatcatttctatgttcgtcattccattacctacctatctggttgatttcctgatcgcactgaatatcgtactggcgatattggtgtttatggggtcgttctacattgacagaatcctcagtttttcaacgtttcctgcggtactgttaattaccacgctctttcgtctggcattatcgatcagtaccagccgtcttatcttgattgaagccgatgccggtgaaattatcgccacgttcgggcaattcgttattggcgatagcctggcggtgggttttgttgtcttctctattgtcaccgtggtccagtttatcgttattaccaaaggttcagaacgcgtcgcggaagtcgcggcccgattttctctggatggtatgcccggtaaacagatgagtattgatgccgatttgaaggccggtattattgatgcggatgctgcgcgcgaacggcgaagcgtactggaaagggaaagccagctttacggttcctttgacggtgcgatgaagtttatcaaaggtgacgctattgccggcatcattattatctttgtgaactttattggcggtatttcggtggggatgacccgccatggtatggatttgtcctccgctctgtctacttataccatgctgaccattggtgatggtcttgtcgcccagatccccgcattgttgattgcgattagtgccggttttatcgtgactcgcgtaaatggcgatagcgataatatggggcggaatatcatgacgcagctgttgaacaacccatttgtattggttgttacggctattttgaccatttcaatgggaactctgccgggattcccgctgccggtatttgttattttatcggtggttttaagcgtactcttctattttaaattccgtgaagcaaaacgtagcgccgccaaacctaaaaccagcaaaggcgagcagccgcttagtattgaggaaaaagaagggtcgtcgttgggactgattggcgatctcgataaagtctctacagagaccgtaccgttgatattacttgtgccgaagagccggcgtgaagatctggaaaaagctcaacttgcggagcgtctacgtagtcagttctttattgattatggcgtgcgcctgccggaagtattgttacgcgatggcgagggcctggacgataacagcatcgtattgttgattaatgagatccgtgttgaacaatttacggtctattttgatttgatgcgagtggtaaattattccgatgaagtcgtgtcctttggtattaatccaacaatccatcagcaaggtagcagtcagtatttctgggtaacgcatgaagagggggagaaactccgggagcttggctatgtgttgcggaacgcgcttgatgagctttaccactgtctggcggtgaccgtggcgcgcaacgtcaatgaatatttcggtattcaggaaacaaaacatatgctggaccaactggaagcgaaatttcctgatttacttaaagaagtgctcagacatgccacggtacaacgtatatctgaagttttgcagcgtttattaagcgaacgtgtttccgtgcgtaatatgaaattaattatggaagcgctcgcattgtgggcgccaagagaaaaagatgtcattaaccttgtagagcatattcgtggagcaatggcgcgttatatttgtcataaattcgccaatggcggcgaattacgagcagtaatggtatctgctgaagttgaggatgttattcgcaaagggatccgtcagacctctggcagtaccttcctcagccttgacccggaagcctccgctaatttgatggatctcattacacttaagttggatgatttattgattgcacataaagatcttgtcctccttacgtctgtcgatgtccgtcgatttattaagaaa

SEQ ID NO: 14 - DNA, 1720 bp, Escherichia coli
cggaaatattgacatgggatgatgaacaatgggaggtatttgtccatgattggcttattgtctgtaaatcagatgattacccgtggagcgaacgtttgggaggagctggagataaaggtagagacgttgttggatataaatcggatcctaacgtagaaggttattcttgggataattatcaatgcaaactgtacaaaaaaagtttagggttctctgatgttgtagttgagtttggaaaacttatctattttactctgaatggtgattatcccatccctcagaagtacttttttgtggcaccctatgatttatctactacattttctaatttattgaaaaataaaaacgagcttaaaaaagcagtccttgattcatgggattcagcaatttcaaaaaaataactaaaaagattgatattccattagatgatgaaataaaaaaatatattgaggattttgattttagtattttttactctctacccttatcattgattttaaatgatattgcaaatacacacctttattttaagtactttaacgagctgtatgtcgtgagaatccctccaaatgaaattccaacatacaattcaaaaaaagagtctgtatatgttaatgcactgcttcaagcctattcagagcatggaaataaaacttatagttctttcttagagcttgatgatccatacagacgacactttaataatagtagaaatgatttttattttgcatcttcgcttgaggtttttgtccgcgaagtatttaaagatgatgtattcaaagcattgaaatgttacatttcatcttcaattgaacccgtcttttatgaagaccataattatgcatttattaggtgtaatgcagtcttgaagcaggctgttctgacaccaattgcacattcagtactatcaaaaatatgtgaagcaaatgataaaaaaggaatatgccatcatttggttaatgatggtgaagtaatttggacggtgagataatggttagaatttataattcaagtttagaagtggcatgtcgaatggcgaaagtgctcgtcgctatttatccttcttcattaagccttgaacggcttatttgttttgattttattttagtaaatcttaaggattttttacctgaagagattagtcttcatcctccaataccccgtagagatgctcagttagccctaaaacgagagattgttttagaatcattggctttgttgcaaggctatgaactagcctcaaaaatttatacacatcgtggttttgtatataaagcttctgaaaaaacatatgcatttacaaattctctacataatgaatatgttgcgcagatggagcataatataaatttggtggttaagttatatagtgatattcctgatgagcagttgcaatcaattataaaaaataaaattggcaaatatgatatggaatttaattatgaatgacaatttttttacgttcagaaaaataaaggtaaccggattcaataaattagatgctataattgaatttggttctaaattgactattttatatggtggttctgactctggaaaaacatacatatattatttgattcgatatttattagggagtgaaaaactaaaaaataaagatatcgatcatgctcaaggttatgatttagcctatctggaatttaattttcaaggtagggtaatgacaattgagaggtctcttcaggatagcgcccattac

(19) United States
(12) Patent Application Publication
(10) Pub. No.: US 2012/0052497 A1
(43) Pub. Date: Mar. 1, 2012

(54) METHOD OF SIMULTANEOUSLY AMPLIFYING TARGET SEQUENCES FROM SALMONELLA SPP. AND E. COLI O157:H7 AND KIT THEREFOR

(75) Inventor: Win Den CHEUNG, Olney, MD (US)

(73) Assignee: SAMSUNG TECHWIN CO., LTD., Kyongsangnam-do (KR)

(21) Appl. No.: 13/159,904

(22) Filed: Jun. 14, 2011

Related U.S. Application Data
(60) Provisional application No. 61/378,060, filed on Aug. 30, 2010.

(51) Int. Cl. C12Q 1/68

(52) U.S. Cl. 435/6.11; 435/6.12

(57) ABSTRACT

Methods are described for the rapid, simultaneous and quantitative PCR detection of pathogenic Salmonella spp. and E. coli O157: H7 nucleic acid sequences in a sample in real-time. The detection method is fast, accurate and suitable for high throughput applications. Convenient, user-friendly and reliable diagnostic kits are also described for the simultaneous detection of Salmonella and E. coli O157: H7 in food samples and on surfaces.

### CROSS-REFERENCE TO RELATED APPLICATIONS

This application claims priority from U.S. Provisional Patent Application No. 61/378,060, filed on Aug. 30, 2010, the contents of which are hereby incorporated by reference in their entirety.

### FIELD

Methods and kits are disclosed for simultaneously amplifying target sequences from Salmonella spp. and E. coli O157: H7 in a sample.

### BACKGROUND

Salmonella, a rod-shaped, Gram-negative Enterobacteria is closely related to the Escherichia genus and can be found worldwide in warm- and cold-blooded animals, including humans. Salmonella causes diseases such as typhoid fever, paratyphoid fever, and the food-borne illness, salmonellosis.

E. coli O157:H7, an enterohemorrhagic strain of Escherichia coli, also causes food-borne illness, resulting in hemorrhagic diarrhea in children and the elderly, which can lead to kidney failure.

Methods of detecting Salmonella spp. and E. coli O157: H7 in samples can be quite cumbersome and time consuming to implement because an initial pre-enrichment is usually required to increase the bacterial concentration (typically 10⁵ CFU/ml) to a level that can be detected by immunoassay.

An increasingly viable alternative to immunoassays are diagnostic kits based on PCR detection of bacterial nucleic acids. Specifically, there is an on-going need for user friendly, accurate kits for the simultaneous PCR detection of Salmonella and E. coli O157: H7 infection.

### SUMMARY

Methods and kits are described for the rapid, simultaneous and quantitative real-time PCR detection of Salmonella and E. coli O157: H7 nucleic acid sequences in a biological sample. The procedure promises to facilitate the high throughput detection of Salmonella spp. and E. coli O157: H7 in a cost effective and reliable manner.

In one embodiment, a method is disclosed for the simultaneous detection of both Salmonella spp. and E. coli O157: H7 in a sample comprising the steps of providing a sample to be tested for the presence of Salmonella spp. and E. coli O157: H7, providing a pair of Salmonella-specific forward and reverse amplification primers that can anneal to a Salmonella-specific target DNA and a pair of E. coli O157: H7-specific forward and reverse amplification primers that can anneal to a E. coli O157: H7-specific target DNA, amplifying a PCR fragment between the first and second Salmonella-specific amplification primers and a PCR fragment between the first and second E. coli O157: H7-specific amplification primers in the presence of an amplifying polymerase activity and amplification buffer, wherein the concentration of the amplifying polymerase is equal to or higher than 0.1 unit/μl, and detecting the Salmonella-specific and E. coli O157: H7-specific PCR amplification products, wherein the detection of PCR amplification products indicates the presence of Salmonella and E. coli O157: H7 in said sample.

The amplifying polymerase can be a thermostable DNA polymerase having a concentration equal to or higher than 0.8 unit/μl or from 0.1 to 1 unit/μl.

The ratio of the number of copies of the Salmonella target nucleic sequence and the number of copies of the E. coli O157: H7 target nucleic sequence in the sample can be equal to or greater than 10:1, or equal to or smaller than 1:10.

In another embodiment, a method is disclosed for the simultaneous detection of both Salmonella spp. and E. coli O157: H7 in a sample comprising the steps of providing a sample to be tested for the presence of Salmonella and E. coli O157: H7, providing a pair of Salmonella-specific forward and reverse amplification primers that can anneal to a Salmonella-specific target DNA and a pair of E. coli O157: H7-specific forward and reverse amplification primers that can anneal to a E. coli O157: H7-specific target DNA, providing a Salmonella-specific probe and an E. coli O157: H7-specific probe, each probe comprising a detectable label and DNA and RNA nucleic acid sequences that are substantially complimentary to either the Salmonella-specific or E. coli O157: H7-specific target DNAs respectively, amplifying a PCR fragment between the Salmonella-specific forward and reverse amplification primers and a PCR fragment between the E. coli O157: H7-specific forward and reverse amplification primers in the presence of an amplifying polymerase activity, amplification buffer; an RNAse H activity and the Salmonella-specific and E. coli O157: H7-specific probes under conditions where the RNA sequences within each probe can form a RNA: DNA heteroduplex with a complimentary target DNA sequence in the PCR fragments, and detecting a real-time increase in the emission of a signal from the label on the Salmonella-specific and E. coli O157: H7-specific probes, wherein the increase in signal indicates the presence of the Salmonella and E. coli O157: H7 in the sample.

In another embodiment, a method is disclosed for the simultaneous detection of both Salmonella spp. and E. coli O157: H7 in a sample comprising the steps of providing a sample to be tested for the presence of Salmonella and E. coli O157: H7 target RNAs, providing a pair of Salmonella-specific forward and reverse amplification primers that can anneal to a Salmonella-specific target DNA and a pair of E. coli O157: H7-specific forward and reverse amplification primers that can anneal to a E. coli O157: H7-specific target DNA, providing a Salmonella-specific probe and an E. coli O157: H7-specific probes, each probe comprising a detectable label and DNA and RNA nucleic acid sequences that are substantially complimentary to either the Salmonella-specific or E. coli O157: H7-specific target DNAs respectively, reverse transcribing the Salmonella-specific and E. coli O157: H7 target RNAs in the presence of a reverse transcriptase activity and the Salmonella-specific reverse amplification primer and E. coli O157: H7-specific reverse amplification primer to produce a Salmonella-specific and E. coli O157: H7-specific target cDNA sequences, amplifying a PCR fragment between the Salmonella-specific forward and reverse amplification primers and a PCR fragment between the E. coli O157: H7-specific forward and reverse amplification primers in the presence of the Salmonella-specific and E. coli O157: H7-specific target cDNA sequences, an amplifying polymerase activity, an amplification buffer; an RNAse H activity, the Salmonella-specific and E. coli O157: H7-specific probes under conditions where the RNA sequences within each of the probes can form a RNA: DNA heteroduplex with complimentary Salmonella-specific and E. coli O157: H7-specific target cDNA sequences; and detecting a real-time increase in the emission of a signal from the label on the Salmonella-specific and E. coli O157: H7-specific probes, wherein the increase in signal indicates the presence of the Salmonella and E. coli O157: H7 in the sample.

The real-time increase in the emission of the signal from the label on the Salmonella-specific and E. coli O157: H7-specific probes can result from the RNAse H cleavage of the RNA: DNA heteroduplex formed between the RNA sequences of the Salmonella-specific probes and one of the strands of the Salmonella-specific target DNA sequences present in the Salmonella-specific PCR fragments and the RNAse H cleavage of the RNA: DNA heteroduplex formed between the RNA sequences of the E. coli O157: H7-specific probes and one of the strands of the E. coli O157: H7-specific target DNA sequences present in the E. coli O157: H7-specific PCR fragments.

The DNA and RNA sequences of the Salmonella-specific and E. coli O157: H7-specific probes can be covalently linked. The probes can be labeled with a fluorescent label or with a FRET pair.

The amplification buffer can be a Tris-acetate buffer.

The PCR fragments can be linked to a solid support.

The amplifying polymerase activity can be an activity of a thermostable DNA polymerase. The RNAse H activity can be the activity of a thermostable RNAse H or hot start RNAse H activity.

The sample can be a food sample or a surface wipe sample.

The nucleic acid within the sample may be pre-treated with uracil-N-glycosylase that is inactivated prior to PCR amplification.

The Salmonella-specific probe can have a structure of R1-X-R2 and the E. coli O157: H7-specific probe can have a structure of R1′-X-R2′, wherein R1, R1′, R2 and R2′ are each selected from the group consisting of a nucleic acid and a nucleic acid analog, and X may be a first RNA, and the R1, R1′, R2 and R2′ each can be coupled to a detectable label.

The pair of Salmonella-specific forward and reverse amplification primers comprises a forward primer (SEQ ID NO: 1) and a reverse primer ((SEQ ID NO: 2), and the pair of E. coli O157: H7-specific amplification forward and reverse primers comprises a forward primer (SEQ ID NO: 3) and a reverse primer ((SEQ ID NO: 4).

The target DNA can be amplified by rolling circle amplification (RCA), nucleic acid sequence based amplification (NASBA), or strand displacement amplification (SDA).

In another embodiment, a kit is described for simultaneously amplifying and detecting target sequences from Salmonella and E. coli O157: H7 in a sample comprising a pair of Salmonella-specific forward and reverse amplification primers, a pair of E. coli O157: H7-specific forward and reverse amplification primers, a Salmonella-specific probe which has a structure of R1-X-R2, an E. coli O157: H7-specific probe which has a structure of R1′-X-R2′, a RNase H, and an amplifying polymerase activity, wherein R1, R1′, R2 and R2′ are each selected from the group consisting of a nucleic acid and a nucleic acid analog, and X may be a first RNA, and the R1, R1′, R2 and R2′ each are coupled to a detectable label.

The amplifying polymerase activity can be a Taq polymerase having a concentration equal to or higher than 0.1 unit/μl.

The pair of Salmonella-specific amplification forward and reverse primers can be a forward primer (SEQ ID NO: 1) and a reverse primer ((SEQ ID NO: 2), and the pair of E. coli O157: H7-specific amplification primers can be a forward primer (SEQ ID NO: 3) and a reverse primer ((SEQ ID NO: 4).

The Salmonella-specific probe can have a nucleotide sequence of SEQ ID NO: 5 and the E. coli O157: H7-specific probe can have a nucleotide sequence of SEQ ID NO: 6.

The kit can also include a reverse transcriptase activity for the reverse transcription of a Salmonella-specific and E. coli O157: H7-specific target RNA sequences to produce Salmonella-specific and E. coli O157: H7-specific target cDNA sequences.

The kit may also have an amplification buffer.

The DNA and RNA sequences of the Salmonella-specific or the E. coli O157: H7-specific probe can be covalently linked.

The Salmonella-specific or the E. coli O157: H7-specific probe can be labeled with a fluorescent compound or with a FRET pair.

The Salmonella-specific and E. coli O157: H7-specific probes may be linked to a solid support.

The amplifying polymerase activity can be an activity of a thermostable DNA polymerase.

The RNAse H activity can be the activity of a thermostable RNAse H or hot start RNAse H activity.

The kit may also include uracil-N-glycosylase or other reagents required for sample preparation.

The previously described embodiments have many advantages, including the ability to detect simultaneously pathogenic Salmonella and E. coli O157: H7 nucleic acid sequences in a sample in real-time. The detection method is fast, accurate and suitable for high throughput applications. Convenient, user-friendly and reliable diagnostic kits are also described for the detection of Salmonella and E. coli O157: H7 in food samples and on surfaces.

### BRIEF DESCRIPTION OF THE DRAWINGS

The skilled artisan will understand that the drawings, described below, are for illustration purposes only. The figures are not intended to limit the scope of the teachings in any way.

FIG. 1 shows real-time polymerization chain reaction (PCR) results when only a Salmonella target sequence exists and when a Salmonella target sequence and an E. coli O157: H7 target sequence coexist at different concentrations.

FIG. 2 shows real-time PCR results when only an E. coli O157: H7 target sequence exists and when a Salmonella target sequence and an E. coli O157: H7 target sequence coexist at different concentrations.

FIG. 3 is a graph of a Cp value with respect to the number of copies of an invasion A (invA) plasmid target when the invA plasmid and an E. coli O157: H7 I fragment exist at seven log concentrations.

FIG. 4 is a graph of a Cp value with respect to the number of copies of an E. coli O157: H7 I fragment when an invA plasmid and the E. coli O157: H7 I fragment exist at seven log concentrations and a low concentration of a DNA Taq polymerase was used.

FIG. 5 is a graph showing an effect of the number of copies of a Salmonella invA plasmid on amplification of an E. coli O157: H7 I fragment, with respect to a DNA Taq polymerase and a low concentration of a DNA Taq polymerase was used.

FIG. 6 is a graph of a Cp value with respect to the number of copies a Salmonella invA plasmid target when a Salmonella invA plasmid and the E. coli O157: H7 I fragment exist at seven log concentrations and a high concentration of a DNA Taq polymerase was used.

FIG. 7 is a graph of a Cp value with respect to the number of copies of an E. coli O157: H7I fragment when a Salmonella invA plasmid and the E. coli O157: H7I fragment exist at seven log concentrations and a high concentration of a DNA Taq polymerase was used.

### DETAILED DESCRIPTION

The practice of the embodiments described herein employs, unless otherwise indicated, conventional molecular biological techniques within the skill of the art. Such techniques are well known to the skilled worker, and are explained fully in the literature. See, e.g., Ausubel, et al., ed., Current Protocols in Molecular Biology, John Wiley & Sons, Inc., NY, N.Y. (1987-2008), including all supplements; Sambrook, et al., Molecular Cloning: A Laboratory Manual, 2nd Edition, Cold Spring Harbor, N.Y. (1989).

Unless defined otherwise, all technical and scientific terms used herein have the same meaning as is commonly understood by one of skill in the art. The specification also provides definitions of terms to help interpret the disclosure and claims of this application. In the event a definition is not consistent with definitions elsewhere, the definition set forth in this application will control.

As used herein, the term “nucleic acid” refers to an oligonucleotide or polynucleotide, wherein said oligonucleotide or polynucleotide may be modified or may comprise modified bases. Oligonucleotides are single-stranded polymers of nucleotides comprising from 2 to 60 nucleotides. Polynucleotides are polymers of nucleotides comprising two or more nucleotides. Polynucleotides may be either double-stranded DNAs, including annealed oligonucleotides wherein the second strand is an oligonucleotide with the reverse complement sequence of the first oligonucleotide, single-stranded nucleic acid polymers comprising deoxythymidine, single-stranded RNAs, double stranded RNAs or RNA/DNA heteroduplexes. Nucleic acids include, but are not limited to, genomic DNA, cDNA, hnRNA, snRNA, mRNA, rRNA, tRNA, fragmented nucleic acid, nucleic acid obtained from subcellular organelles such as mitochondria or chloroplasts, and nucleic acid obtained from microorganisms or DNA or RNA viruses that may be present on or in a biological sample. Nucleic acids may be composed of a single type of sugar moiety, e.g., as in the case of RNA and DNA, or mixtures of different sugar moieties, e.g., as in the case of RNA/DNA chimeras.

A “target DNA or “target RNA”” or “target nucleic acid,” or “target nucleic acid sequence” refers to a nucleic acid that is targeted by DNA amplification. A target nucleic acid sequence serves as a template for amplification in a PCR reaction or reverse transcriptase-PCR reaction. Target nucleic acid sequences may include both naturally occurring and synthetic molecules. Exemplary target nucleic acid sequences include, but are not limited to, genomic DNA or genomic RNA.

The term “nucleic acid analog,” as used herein, refers to a molecule including one or more nucleotide analogs and/or one or more phosphate ester analogs and/or one or more pentose analogs. An example of the nucleic acid analog is a molecule in which a phosphate ester bond and/or a sugar phosphate ester bond is to be substituted with another type of bond, for example, an N-(2-aminoethyl)-glycine amide bond and other amide bonds. Another example of the nucleic acid analog may be a molecule that includes one or more nucleotide analogs and/or one or more phosphate ester analogs and/or one or more pentose analogs and forms a double bond by hybridization.

The terms “annealing” and “hybridization” used herein are interchangeably used with each other, and refer to a base-pairing interaction for allowing formation of a double-strand, a triple-strand, or a more than triple-strand between one nucleic acid and another nucleic acid. An example of the base-pairing interaction may be a base specific primary interaction by a Watson/Crick and Hoogsteen-type hydrogen bond, for example, A/T, and a G/C interaction. In addition, base-stacking and a hydrophobic bond may also contribute to double-strand stability.

As used herein, “label” or “detectable label” can refer to any chemical moiety attached to a nucleotide, nucleotide polymer, or nucleic acid binding factor, wherein the attachment may be covalent or non-covalent. Preferably, the label is detectable and renders said nucleotide or nucleotide polymer detectable to the practitioner of the invention. Detectable labels can include luminescent molecules, chemiluminescent molecules, fluorochromes, fluorescent quenching agents, colored molecules, radioisotopes or scintillants. Detectable labels can also include any useful linker molecule (such as biotin, avidin, streptavidin, HRP, protein A, protein G, antibodies or fragments thereof, Grb2, polyhistidine, Ni²⁺, FLAG tags, myc tags), heavy metals, enzymes (examples include alkaline phosphatase, peroxidase and luciferase), electron donors/acceptors, acridinium esters, dyes and calorimetric substrates. It is also envisioned that a change in mass may be considered a detectable label, as is the case of surface plasmon resonance detection. The skilled artisan would readily recognize useful detectable labels that are not mentioned above, which may be employed in the operation of the present invention.

### Selection of Primer Sequences

Primer pairs are selected according to their ability not to form primer dimers during PCR amplification. Such primers are capable of detecting single target molecules in as little as about 40 PCR cycles using optimum amplification conditions.

A “primer dimer” is a potential by-product in PCR that consists of primer molecules that have partially hybridized to each other because of strings of complementary bases in the primers. As a result, the DNA polymerase amplifies the primer dimer, leading to competition for PCR reagents, thus potentially inhibiting amplification of the DNA sequence targeted for PCR amplification. In real-time PCR, primer dimers may interfere with accurate quantification by reducing sensitivity.

A Salmonella nucleic acid sequence targeted for DNA amplification is first selected from Salmonella nucleic sequences known in the art. As used herein, the term “Salmonella target sequence” refers to a DNA or RNA sequence comprising the nucleic acid sequence of a bacterium of the genus Salmonella. It includes but is not limited to, species S. enterica and S. bongori that include, but are not limited to, the subspecies: enterica (I), salamae (II), arizonae (Ma), diarizonae (IIIb), houtenae (IV), and indica (VI). Exemplary serogroups and serovars of the subspecies Salmonella enterica can be found in the U.S. Pat. No. 7,659,381, which is incorporated herein by reference in its entirety.

Exemplary Salmonella nucleic acid sequences that may be targeted for amplification according to the present invention are taught by the following publications: Liu W Q et al., “Salmonella paratyphi C: genetic divergence from Salmonella choleraesuis and pathogenic convergence with Salmonella typhi”, PLoS One, 2009; 4(2):e4510; Thomson N R et al., “Comparative genome analysis of Salmonella enteritidis PT4 and Salmonella gallinarum 287/91 provides insights into evolutionary and host adaptation pathways,” Genome Res, 2008 October; 18(10): 1624-37; Encheva V et al., “Proteome analysis of serovars typhimurium and Pullorum of Salmonella enterica subspecies I.”, BMC Microbiol, 2005 Jul. 18; 5:42; McClelland M et al., “Comparison of genome degradation in Paratyphi A and Typhi, human-restricted serovars of Salmonella enterica that cause typhoid”, Nat Genet, 2004 December; 36(12):1268-74; Chiu C H et al., “Salmonella enterica serotype Choleraesuis: epidemiology, pathogenesis, clinical disease, and treatment,” Clin Microbiol Rev, 2004 April; 17(2):311-22; Deng W et al., “Comparative genomics of Salmonella enterica serovar Typhi strains Ty2 and CT18,” J Bacteriol, 2003 April; 185(7):2330-7; Parkhill J et al., “Complete genome sequence of a multiple drug resistant Salmonella enterica serovar Typhi CT18.”, Nature, 2001 Oct. 25; 413(6858):848-52; McClelland M et al., “Complete genome sequence of Salmonella enterica serovar typhimurium LT2,” Nature, 2001 Oct. 25; 413(6858):852-6, of which contents are incorporated herein by reference. An exemplary nucleotide sequence of the complete 4857432 bp genome of Salmonella enterica subsp. enterica serovar typhimurium str. LT2 is available under Genbank Accession No. NC_{—}003197.

In an embodiment, the amplification probe which anneals to the target Salmonella invA nucleic acid sequence may be:

| Salmonella-Forward primer: | (SEQ ID NO: 1) |
|---|---|
| 5′-TCGTCATTCCATTACCTACC, | |
| Salmonella-Reverse primer: | (SEQ ID NO: 2) |

5′-TACTGATCGATAATGCCAGACGAA.

In another embodiment, the target nucleic acid sequence is the Salmonella-specific InvA gene nucleic acid sequence having the following DNA sequence.

SEQ ID NO: 13, Salmonella enterica InvA gene (GenBank Accession No.: U43272.1):
AACAGTGCTCGTTTACGACCTGAATTACTGATTCTGGTACTAATGGTGATGATCATTTCT
ATGTTCGTCATTCCATTACCTACCTATCTGGTTGATTTCCTGATCGCACTGAATATCGTA
CTGGCGATATTGGTGTTTATGGGGTCGTTCTACATTGACAGAATCCTCAGTTTTTCAACG
TTTCCTGCGGTACTGTTAATTACCACGCTCTTTCGTCTGGCATTATCGATCAGTACCAGC
CGTCTTATCTTGATTGAAGCCGATGCCGGTGAAATTATCGCCACGTTCGGGCAATTCGTT
ATTGGCGATAGCCTGGCGGTGGGTTTTGTTGTCTTCTCTATTGTCACCGTGGTCCAGTTT
ATCGTTATTACCAAAGGTTCAGAACGCGTCGCGGAAGTCGCGGCCCGATTTTCTCTGGAT
GGTATGCCCGGTAAACAGATGAGTATTGATGCCGATTTGAAGGCCGGTATTATTGATGCG
GATGCTGCGCGCGAACGGCGAAGCGTACTGGAAAGGGAAAGCCAGCTTTACGGTTCCTTT
GACGGTGCGATGAAGTTTATCAAAGGTGACGCTATTGCCGGCATCATTATTATCTTTGTG
AACTTTATTGGCGGTATTTCGGTGGGGATGACCCGCCATGGTATGGATTTGTCCTCCGCT
CTGTCTACTTATACCATGCTGACCATTGGTGATGGTCTTGTCGCCCAGATCCCCGCATTG
TTGATTGCGATTAGTGCCGGTTTTATCGTGACTCGCGTAAATGGCGATAGCGATAATATG
GGGCGGAATATCATGACGCAGCTGTTGAACAACCCATTTGTATTGGTTGTTACGGCTATT
TTGACCATTTCAATGGGAACTCTGCCGGGATTCCCGCTGCCGGTATTTGTTATTTTATCG
GTGGTTTTAAGCGTACTCTTCTATTTTAAATTCCGTGAAGCAAAACGTAGCGCCGCCAAA
CCTAAAACCAGCAAAGGCGAGCAGCCGCTTAGTATTGAGGAAAAAGAAGGGTCGTCGTTG
GGACTGATTGGCGATCTCGATAAAGTCTCTACAGAGACCGTACCGTTGATATTACTTGTG
CCGAAGAGCCGGCGTGAAGATCTGGAAAAAGCTCAACTTGCGGAGCGTCTACGTAGTCAG
TTCTTTATTGATTATGGCGTGCGCCTGCCGGAAGTATTGTTACGCGATGGCGAGGGCCTG
GACGATAACAGCATCGTATTGTTGATTAATGAGATCCGTGTTGAACAATTTACGGTCTAT
TTTGATTTGATGCGAGTGGTAAATTATTCCGATGAAGTCGTGTCCTTTGGTATTAATCCA
ACAATCCATCAGCAAGGTAGCAGTCAGTATTTCTGGGTAACGCATGAAGAGGGGGAGAAA
CTCCGGGAGCTTGGCTATGTGTTGCGGAACGCGCTTGATGAGCTTTACCACTGTCTGGCG
GTGACCGTGGCGCGCAACGTCAATGAATATTTCGGTATTCAGGAAACAAAACATATGCTG
GACCAACTGGAAGCGAAATTTCCTGATTTACTTAAAGAAGTGCTCAGACATGCCACGGTA
CAACGTATATCTGAAGTTTTGCAGCGTTTATTAAGCGAACGTGTTTCCGTGCGTAATATG
AAATTAATTATGGAAGCGCTCGCATTGTGGGCGCCAAGAGAAAAAGATGTCATTAACCTT
GTAGAGCATATTCGTGGAGCAATGGCGCGTTATATTTGTCATAAATTCGCCAATGGCGGC
GAATTACGAGCAGTAATGGTATCTGCTGAAGTTGAGGATGTTATTCGCAAAGGGATCCGT
CAGACCTCTGGCAGTACCTTCCTCAGCCTTGACCCGGAAGCCTCCGCTAATTTGATGGAT
CTCATTACACTTAAGTTGGATGATTTATTGATTGCACATAAAGATCTTGTCCTCCTTACG
TCTGTCGATGTCCGTCGATTTATTAAGAAA.

As used herein, the term “oligonucleotide” is used sometimes interchangeably with “primer” or “polynucleotide.” The term “primer” refers to an oligonucleotide that acts as a point of initiation of DNA synthesis in a PCR reaction. A primer is usually about 15 to about 35 nucleotides in length and hybridizes to a region complementary to the target sequence.

Oligonucleotides may be synthesized and prepared by any suitable methods (such as chemical synthesis), which are known in the art. Oligonucleotides may also be conveniently available through commercial sources.

Exemplary E. coli O157:H7 nucleic acid sequences that may be targeted for amplification according to the present invention are taught by the following publications: Ogura Y et al., “Extensive genomic diversity and selective conservation of virulence-determinants in enterohemorrhagic Escherichia coli strains of O157 and non-O157 serotypes,” Genome Biol, 2007; 8(7):R138; Steele M et al., “Identification of Escherichia coli O157:H7 genomic regions conserved in strains with a genotype associated with human infection,” Appl Environ Microbiol, 2007 January; 73(1)22-31; Ohnishi M et al., “Genomic diversity of enterohemorrhagic Escherichia coli O157 revealed by whole genome PCR scanning,” Proc Natl Acad Sci USA, 2002 Dec. 24; 99(26)17043-8; Schneider D et al., “Genomic comparisons among Escherichia coli strains B, K-12, and O157:H7 using IS elements as molecular markers,” BMC Microbiol, 2002 Jul. 9; 2:18; Lim A et al., “Shotgun optical maps of the whole Escherichia coli O157:H7 genome,” Genome Res, 2001 September; 11(9):1584-93; Hayashi T et al., “Complete genome sequence of enterohemorrhagic Esherichia coli O157:H7 and genomic comparison with a laboratory strain K-12,” DNA Res, 2001 Feb. 28; 8(1):11-22; Perna N T et al., “Genome sequence of enterohaemorrhagic Escherichia coli O157:H7,” Nature, 2001 Jan. 25; 409(6819):529-33, of which contents are incorporated herein by reference. An exemplary nucleotide sequence of the complete 5528445 bp genome of Escherichia coli O157:H7 str. EDL933 is available under Genbank Accession No. AE005174.

The primer specific to E. coli O157:H7 may be specific to an E. coli O157:H7 I fragment. For example, the primer specific to E. coli O157:H7 may include an E. coli O157 I-F1 primer and an O157 I-R primer:

| | |
|---|---|
| O157 I-FI: | (SEQ ID NO: 3) |

5′-AAC GAG CTG TAT GTC GTG AGA ATC-3′,

| | |
|---|---|
| O157 I-R: | (SEQ ID NO: 4) |

5′-ATG GAT CAT CAA GCT CTA AGA AAG AAC-3′.

In another embodiment, the target nucleic acid sequence is the E. coli O157:H7-specific I fragment nucleic acid sequence having the following DNA sequence.

SEQ ID NO: 14, Escherichia coli O157: H7 I fragment:
CGGAAATATTGACATGGGATGATGAACAATGGGAGGTATTTGTCCATGATTGGCTTATTG
TCTGTAAATCAGATGATTACCCGTGGAGCGAACGTTTGGGAGGAGCTGGAGATAAAGGTA
GAGACGTTGTTGGATATAAATCGGATCCTAACGTAGAAGGTTATTCTTGGGATAATTATC
AATGCAAACTGTACAAAAAAAGTTTAGGGTTCTCTGATGTTGTAGTTGAGTTTGGAAAAC
TTATCTATTTTACTCTGAATGGTGATTATCCCATCCCTCAGAAGTACTTTTTTGTGGCAC
CCTATGATTTATCTACTACATTTTCTAATTTATTGAAAAATAAAAACGAGCTTAAAAAAG
CAGTCCTTGATTCATGGGATTCAGCAATTTCAAAAAAATAACTAAAAAGATTGATATTCC
ATTAGATGATGAAATAAAAAAATATATTGAGGATTTTGATTTTAGTATTTTTTACTCTCT
ACCCTTATCATTGATTTTAAATGATATTGCAAATACACACCTTTATTTTAAGTACTTTAA
CGAGCTGTATGTCGTGAGAATCCCTCCAAATGAAATTCCAACATACAATTCAAAAAAAGA
GTCTGTATATGTTAATGCACTGCTTCAAGCCTATTCAGAGCATGGAAATAAAACTTATAG
TTCTTTCTTAGAGCTTGATGATCCATACAGACGACACTTTAATAATAGTAGAAATGATTT
TTATTTTGCATCTTCGCTTGAGGTTTTTGTCCGCGAAGTATTTAAAGATGATGTATTCAA
AGCATTGAAATGTTACATTTCATCTTCAATTGAACCCGTCTTTTATGAAGACCATAATTA
TGCATTTATTAGGTGTAATGCAGTCTTGAAGCAGGCTGTTCTGACACCAATTGCACATTC
AGTACTATCAAAAATATGTGAAGCAAATGATAAAAAAGGAATATGCCATCATTTGGTTAA
TGATGGTGAAGTAATTTGGACGGTGAGATAATGGTTAGAATTTATAATTCAAGTTTAGAA
GTGGCATGTCGAATGGCGAAAGTGCTCGTCGCTATTTATCCTTCTTCATTAAGCCTTGAA
CGGCTTATTTGTTTTGATTTTATTTTAGTAAATCTTAAGGATTTTTTACCTGAAGAGATT
AGTCTTCATCCTCCAATACCCCGTAGAGATGCTCAGTTAGCCCTAAAACGAGAGATTGTT
TTAGAATCATTGGCTTTGTTGCAAGGCTATGAACTAGCCTCAAAAATTTATACACATCGT
GGTTTTGTATATAAAGCTTCTGAAAAAACATATGCATTTACAAATTCTCTACATAATGAA
TATGTTGCGCAGATGGAGCATAATATAAATTTGGTGGTTAAGTTATATAGTGATATTCCT
GATGAGCAGTTGCAATCAATTATAAAAAATAAAATTGGCAAATATGATATGGAATTTAAT
TATGAATGACAATTTTTTTACGTTCAGAAAAATAAAGGTAACCGGATTCAATAAATTAGA
TGCTATAATTGAATTTGGTTCTAAATTGACTATTTTATATGGTGGTTCTGACTCTGGAAA
AACATACATATATTATTTGATTCGATATTTATTAGGGAGTGAAAAACTAAAAAATAAAGA
TATCGATCATGCTCAAGGTTATGATTTAGCCTATCTGGAATTTAATTTTCAAGGTAGGGT
AATGACAATTGAGAGGTCTCTTCAGGATAGCGCCCATTAC

A person of skill in the art will know how to design PCR primers flanking a Salmonella and E. coli O157: H7 genomic sequence of interest. Synthesized oligos are typically between 20 and 26 base pairs in length with a melting temperature, T_{M} of around 55 degrees.

### Enrichment for Bacterial Nucleic Acid Sequences in a Test Sample

Because Salmonella and E. coli O157: H7 require similar growth and nutrition conditions. Salmonella and E. coli O157: H7 all grow well in a similar medium at a temperature of 35° C. to 42° C., and also have a similar doubling time. Thus, Salmonella and E. coli O157: H7 in a sample optionally may be enriched by culture prior to processing and real-time PCR amplification and detection.

An exemplary protocol for detecting target Salmonella and E. coli O157: H7 sequences may include the steps of providing a food sample or surface wipe, mixing the sample or wipe with a growth medium and incubating to increase the number or population of Salmonella and E. coli O157: H7 (“enrichment”), disintegrating Salmonella and E. coli O157: H7 cells (“lysis”), and subjecting the obtained lysate to amplification and detection of target Salmonella and E. coli O157: H7 nucleic acid sequences. Food samples may include, but are not limited to, fish such as salmon, dairy products such as milk, and eggs, poultry, fruit juices, meats such as ground pork, pork, ground beef, or beef, vegetables such as spinach or alfalfa sprouts, or processed nuts such as peanut butter.

The limit of detection (LOD) for food contaminants is described in terms of the number of colony forming units (CFU) that can be detected in either 25 grams of solid or 25 mL of liquid food or on a surface of defined area. By definition, a colony-forming unit is a measure of viable bacterial numbers. Unlike indirect microscopic counts where all cells, dead and living, are counted, CFU measures viable cells. One CFU (one bacterial cell) will grow to form a single colony on an agar plate under permissive conditions. The United States Food Testing Inspection Service defines the minimum LOD as 1 CFU/25 grams of solid food or 25 mL of liquid food or 1 CFU/surface area.

In practice, it is impossible to reproducibly inoculate a food sample or surface with a single CFU and insure that the bacterium survives the enrichment process. This problem is overcome by inoculating the sample at either one or several target levels and analyzing the results using a statistical estimate of the contamination called the Most Probable Number (MPN). As an example, Salmonella and E. coli O157: H7 cultures can be grown to a specific cell density by measuring the absorbance in a spectrophotometer. Ten-fold serial dilutions of the target are plated on agar media and the numbers of viable bacteria are counted. This data is used to construct a standard curve that relates CFU/volume plated to cell density. For the MPN to be meaningful, test samples at several inoculum levels are analyzed. After enrichment and extraction, a small volume of sample is removed for real-time analysis. The ultimate goal is to achieve a fractional recovery of between 25% and 75% (i.e. between 25% and 75% of the samples test positive in the assay using-real-time PCR employing a CataCleave probe, which will be explained below). The reason for choosing these fractional recovery percentages is that they convert to MPN values of between 0.3 CFU and 1.375 CFU for 25 gram samples of solid food, 25 mL samples of liquid food, or a defined area for surfaces. These MPN values bracket the required LOD of 1 CFU/sample. With practice, it is possible to estimate the volume of diluted inoculum (based on the standard curve) to achieve these fractional recoveries.

### Nucleic Acid Template Preparation

In some embodiments, the sample comprises a purified nucleic acid template (e.g., mRNA, rRNA, and mixtures thereof). Procedures for the extraction and purification of RNA from samples are well known in the art. For example, RNA can be isolated from cells using the TRIzol™ reagent (Invitrogen) extraction method. RNA quantity and quality is then determined using, for example, a Nanodrop™ spectrophotometer and an Agilent 2100 bioanalyzer.

In other embodiments, the sample is a cell lysate that is produced by lysing cells using a lysis buffer having a pH of about 6 to about 9, a zwitterionic detergent at a concentration of about 0.125% to about 2%, an azide at a concentration of about 0.3 to about 2.5 mg/ml and a protease such as proteinase K (about 1 mg/ml). After incubation at 55° C. for 15 minutes, the proteinase K is inactivated at 95° C. for 10 minutes to produce a “substantially protein free” lysate that is compatible with high efficiency PCR or reverse transcription PCR analysis.

In one embodiment, the 1× lysis reagent contains 12.5 mM Tris acetate or Tris-HCl or HEPES (4-(2-hydroxyethyl)-1-piperazineethanesulfonic acid) (pH=7-8), 0.25% (w/v) CHAPS, 0.3125 mg/ml sodium azide and proteinase K at 1 mg/ml.

The term “lysate” as used herein, refers to a liquid phase with lysed cell debris and nucleic acids.

As used herein, the term “substantially protein free” refers to a lysate where most proteins are inactivated by proteolytic cleavage by a protease. Protease may include proteinase K. Addition of proteinase K during cell lysis rapidly inactivates nucleases that might otherwise degrade the target nucleic acids. The “substantially protein free” lysate may be or may not be subjected to a treatment to remove inactivated proteins.

For the lysis of gram negative bacteria, such as Salmonella and E. coli, proteinase K to 1 mg/ml may be added to the lysis reagent. After incubation at 55° C. for 15 minutes, the proteinase K is inactivated at 95° C. for 10 minutes to produce a substantially protein free lysate that is compatible with high efficiency PCR or reverse transcription PCR analysis.

As used herein, “zwitterionic detergent” refers to detergents exhibiting zwitterionic character (e.g., does not possess a net charge, lacks conductivity and electrophoretic mobility, does not bind ion-exchange resins, breaks protein-protein interactions), including, but not limited to, CHAPS, CHAPSO and betaine derivatives, e.g. preferably sulfobetaines sold under the brand names Zwittergent® (Calbiochem, San Diego, Calif.) and Anzergent® (Anatrace, Inc. Maumee, Ohio).

In one embodiment, the zwitterionic detergent is CHAPS (CAS Number: 75621-03-3; available from SIGMA-ALDRICH product no. C3023-1G), an abbreviation for 3-[(3-cholamidopropyl)dimethylammonio]-1-propanesulfonate (described in further detail in U.S. Pat. No. 4,372,888) having the structure:

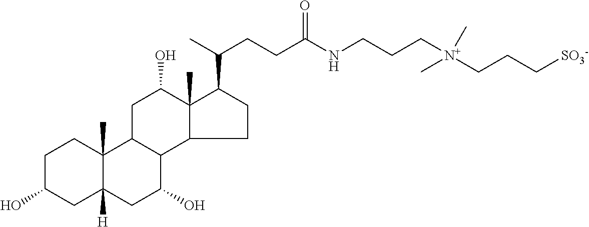

In a further embodiment, CHAPS is present at a concentration of about 0.125% to about 2% weight/volume (w/v) of the total composition. In a further embodiment, CHAPS is present at a concentration of about 0.25% to about 1% w/v of the total composition. In yet another embodiment, CHAPS is present at a concentration of about 0.4% to about 0.7% w/v of the total composition.

In other embodiments, the lysis buffer may include other non-ionic detergents such as Nonidet, Tween or Triton X-100.

As used herein, the term “lysis buffer” refers to a composition that can effectively maintain the pH value between 6 and 9, with a pKa at 25° C. of about 6 to about 9. The buffer described herein is generally a physiologically compatible buffer that is compatible with the function of enzyme activities and enables biological macromolecules to retain their normal physiological and biochemical functions.

Examples of buffers added to a lysis buffer include, but are not limited to, HEPES ((4-(2-hydroxyethyl)-1-piperazineethanesulfonic acid), MOPS (3-(N-morpholino)-propanesulfonic acid), N-tris(hydroxymethyl)methylglycine acid (Tricine), tris(hydroxymethyl)methylamine acid (Tris), piperazine-N,N′-bis(2-ethanesulfonic acid) (PIPES) and acetate or phosphate containing buffers (K2HPO4, KH2PO4, Na2HPO4, NaH2PO4) and the like.

The term “azide” as used herein is represented by the formula —N3. In one embodiment, the azide is sodium azide NaN3 (CAS number 26628-22-8; available from SIGMA-ALDRICH Product number: S2002-25G) that acts as a general bacterioside.

The term “protease,” as used herein, is an enzyme that hydrolyses peptide bonds (has protease activity). Proteases are also called, e.g., peptidases, proteinases, peptide hydrolases, or proteolytic enzymes. The proteases for use according to the invention can be of the endo-type that act internally in polypeptide chains (endopeptidases). In one embodiment, the protease can be the serine protease, proteinase K (EC 3.4.21.64; available from Roche Applied Sciences, recombinant proteinase K 50 U/ml (from Pichia pastoris) Cat. No. 03 115 887 001).

Proteinase K is used to digest protein and remove contamination from preparations of nucleic acid. Addition of proteinase K to nucleic acid preparations rapidly inactivates nucleases that might otherwise degrade the DNA or RNA during purification. It is highly-suited to this application since the enzyme is active in the presence of chemicals that denature proteins and it can be inactivated at temperatures of about 95° C. for about 10 minutes.

In addition to or in lieu of proteinase K, the lysis reagent can comprise a serine protease such as trypsin, chymotrypsin, elastase, subtilisin, streptogrisin, thermitase, aqualysin, plasmin, cucumisin, or carboxypeptidase A, D, C, or Y. In addition to a serine protease, the lysis solution can comprise a cysteine protease such as papain, calpain, or clostripain; an acid protease such as pepsin, chymosin, or cathepsin; or a metalloprotease such as pronase, thermolysin, collagenase, dispase, an aminopeptidase or carboxypeptidase A, B, E/H, M, T, or U. Proteinase K is stable over a wide pH range (pH 4.0-10.0) and is stable in buffers with zwitterionic detergents.

### PCR Amplification of Target Nucleic Acid Sequences

Once the primers are prepared, nucleic acid amplification can be accomplished by a variety of methods, including, but not limited to, the polymerase chain reaction (PCR), nucleic acid sequence based amplification (NASBA), ligase chain reaction (LCR), and rolling circle amplification (RCA). The polymerase chain reaction (PCR) is the method most commonly used to amplify specific target DNA sequences.

“Polymerase chain reaction,” or “PCR,” generally refers to a method for amplification of a desired nucleotide sequence in vitro. Generally, the PCR process consists of introducing a molar excess of two or more extendable oligonucleotide primers to a reaction mixture comprising a sample having the desired target sequence(s), where the primers are complementary to opposite strands of the double stranded target sequence. The reaction mixture is subjected to a program of thermal cycling in the presence of a DNA polymerase, resulting in the amplification of the desired target sequence flanked by the DNA primers.

The technique of PCR is described in numerous publications, including, PCR: A Practical Approach, M. J. McPherson, et al., IRL Press (1991), PCR Protocols: A Guide to Methods and Applications, by Innis, et al., Academic Press (1990), and PCR Technology: Principals and Applications for DNA Amplification, H. A. Erlich, Stockton Press (1989). PCR is also described in many U.S. patents, including U.S. Pat. Nos. 4,683,195; 4,683,202; 4,800,159; 4,965,188; 4,889,818; 5,075,216; 5,079,352; 5,104,792; 5,023,171; 5,091,310; and 5,066,584, each of which is herein incorporated by reference.

The term “sample” refers to any substance containing nucleic acid material.

As used herein, the term “PCR fragment” or “reverse transcriptase-PCR fragment” or “amplicon” refers to a polynucleotide molecule (or collectively the plurality of molecules) produced following the amplification of a particular target nucleic acid. An PCR fragment is typically, but not exclusively, a DNA PCR fragment. A PCR fragment can be single-stranded or double-stranded, or in a mixture thereof in any concentration ratio. A PCR fragment or RT-PCR fragment can be about 100 to about 500 nt or more in length.

A “buffer” is a compound added to an amplification reaction which modifies the stability, activity, and/or longevity of one or more components of the amplification reaction by regulating the pH of the amplification reaction. The buffering agents of the invention are compatible with PCR amplification and site-specific RNase H cleavage activity. Certain buffering agents are well known in the art and include, but are not limited to, Tris, Tricine, MOPS (3-(N-morpholino)propanesulfonic acid), and HEPES (4-(2-hydroxyethyl)-1-piperazineethanesulfonic acid). In addition, PCR buffers may generally contain up to about 70 mM KCl and about 1.5 mM or higher MgCl₂, to about 50-200 μM each of nucleotides dATP, dCTP, dGTP and dTTP. The buffers of the invention may contain additives to optimize efficient reverse transcriptase-PCR or PCR reaction.

The term “nucleotide,” as used herein, refers to a compound comprising a nucleotide base linked to the C-1′ carbon of a sugar, such as ribose, arabinose, xylose, and pyranose, and sugar analogs thereof. The term “nucleotide” includes a ribonucleoside triphosphate such as rATP, rCTP, rGTP, or rUTP, and a deoxyribonucleoside triphosphate such as dATP, dCTP, dGTP, or dTTP.

The term “nucleoside” used herein refers to a combination of a base and a sugar, that is, a nucleotide that does not include a phosphate moiety. The term “nucleoside” and the term “nucleotide” may also be used inter-changeably in the art. For example, dUTP is deoxyribonucleoside triphosphate, and when inserted into DNA, may act as a DNA monomer, that is, dUMP or deoxyuridin monophosphate. In this regard, even when obtained DNA does not include dUTP, it can be said that dUTP is inserted into DNA.

The term nucleotide also encompasses nucleotide analogs. The sugar may be substituted or unsubstituted. Substituted ribose sugars include, but are not limited to, those riboses in which one or more of the carbon atoms, for example the 2′-carbon atom, is substituted with one or more of the same or different Cl, F, —R, —OR, —NR₂ or halogen groups, where each R is independently H, C1-C6 alkyl or C5-C14 aryl. Exemplary riboses include, but are not limited to, 2′-(C1-C6)alkoxyribose, 2′-(C5-C14)aryloxyribose, 2′,3′-didehydroribose, 2′-deoxy-3′-haloribose, 2′-deoxy-3′-fluororibose, 2′-deoxy-3′-chlororibose, 2′-deoxy-3′-aminoribose, 2′-deoxy-3′-(C1-C6)alkylribose, 2′-deoxy-3′-(C1-C6)alkoxyribose and 2′-deoxy-3′-(C5-C14)aryloxyribose, ribose, 2′-deoxyribose, 2′,3′-dideoxyribose, 2′-haloribose, 2′-fluororibose, 2′-chlororibose, and 2′-alkylribose, e.g., 2′-O-methyl, 4′-α-anomeric nucleotides, 1′-α-anomeric nucleotides, 2′-4′- and 3′-4′-linked and other “locked” or “LNA”, bicyclic sugar modifications (see, e.g., PCT published application nos. WO 98/22489, WO 98/39352, and WO 99/14226; and U.S. Pat. Nos. 6,268,490 and 6,794,499).

An additive is a compound added to a composition which modifies the stability, activity, and/or longevity of one or more components of the composition. In certain embodiments, the composition is an amplification reaction composition. In certain embodiments, an additive inactivates contaminant enzymes, stabilizes protein folding, and/or decreases aggregation. Exemplary additives that may be included in an amplification reaction include, but are not limited to, betaine, formamide, KCl, CaCl₂, MgOAc, MgCl₂, NaCl, NH₄OAc, NaI, Na(CO₃)₂, LiCl, MnOAc, NMP, trehalose, demethylsulfoxide (“DMSO”), glycerol, ethylene glycol, dithiothreitol (“DTT”), pyrophosphatase (including, but not limited to Thermoplasma acidophilum inorganic pyrophosphatase (“TAP”)), bovine serum albumin (“BSA”), propylene glycol, glycinamide, CHES, Percoll, aurintricarboxylic acid, Tween 20, Tween 21, Tween 40, Tween 60, Tween 85, Brij 30, NP-40, Triton X-100, CHAPS, CHAPSO, Mackernium, LDAO (N-dodecyl-N,N-dimethylamine-N-oxide), Zwittergent 3-10, Xwittergent 3-14, Xwittergent SB 3-16, Empigen, NDSB-20, T4G32, E. Coli SSB, RecA, nicking endonucleases, 7-deazaG, dUTP, UNG, anionic detergents, cationic detergents, non-ionic detergents, zwittergent, sterol, osmolytes, cations, and any other chemical, protein, or cofactor that may alter the efficiency of amplification. In certain embodiments, two or more additives are included in an amplification reaction. According to the invention, additives may be added to improve selectivity of primer annealing provided the additives do not interfere with the activity of RNase H.

As used herein, the term “thermostable,” as applied to an enzyme, refers to an enzyme that retains its biological activity at elevated temperatures (e.g., at 55° C. or higher), or retains its biological activity following repeated cycles of heating and cooling. Thermostable polynucleotide polymerases find particular use in PCR amplification reactions.

As used herein, an “amplifying polymerase activity” refers to an enzymatic activity that catalyzes the polymerization of deoxyribonucleotides. Generally, the enzyme will initiate synthesis at the 3′-end of the primer annealed to a nucleic acid template sequence, and will proceed toward the 5′ end of the template strand. In certain embodiments, an “amplifying polymerase activity” is a thermostable DNA polymerase.

As used herein, a thermostable polymerase is an enzyme that is relatively stable to heat and eliminates the need to add enzyme prior to each PCR cycle.

Non-limiting examples of thermostable DNA polymerases may include, but are not limited to, polymerases isolated from the thermophilic bacteria Thermus aquaticus (Taq polymerase), Thermus thermophilus (Tth polymerase), Thermococcus litoralis (Tli or VENT™ polymerase), Pyrococcus furiosus (Pfu or DEEPVENT™ polymerase), Pyrococcus woosii (Pwo polymerase) and other Pyrococcus species, Bacillus stearothermophilus (Bst polymerase), Sulfolobus acidocaldarius (Sac polymerase), Thermoplasma acidophilum (Tac polymerase), Thermus rubber (Tru polymerase), Thermus brockianus (DYNAZYME™ polymerase) i (Tne polymerase), Thermotoga maritime (Tma) and other species of the Thermotoga genus (Tsp polymerase), and Methanobacterium thermoautotrophicum (Mth polymerase). The PCR reaction may contain more than one thermostable polymerase enzyme with complementary properties leading to more efficient amplification of target sequences. For example, a nucleotide polymerase with high processivity (the ability to copy large nucleotide segments) may be complemented with another nucleotide polymerase with proofreading capabilities (the ability to correct mistakes during elongation of target nucleic acid sequence), thus creating a PCR reaction that can copy a long target sequence with high fidelity. The thermostable polymerase may be used in its wild type form. Alternatively, the polymerase may be modified to contain a fragment of the enzyme or to contain a mutation that provides beneficial properties to facilitate the PCR reaction. In one embodiment, the thermostable polymerase may be Taq polymerase. Many variants of Taq polymerase with enhanced properties are known and include, but are not limited to, AmpliTaq™, AmpliTaq™, Stoffel fragment, SuperTaq™, SuperTaq™ plus, LA Taq™, LApro Taq™, and EX Taq™. In another embodiment, the thermostable polymerase used in the multiplex amplification reaction of the invention is the AmpliTaq Stoffel fragment.

The nucleic acid polymerase may have a concentration of 0.1 unit/μL or more in a reaction mixture. For example, the concentration of the nucleic acid polymerase in the reaction mixture may be in the range of 0.1 to 10 unit/μL, 0.1 to 5 unit/μL, 0.1 to 2.5 unit/μL, or 0.1 to about 1 unit/μL.

The term “simultaneously” used herein does not necessarily mean the same time, and may also refer to a case of employing a single process or step to detect all of two or more distinct strains or species. For example, the steps may be performed in a single PCR.

In one embodiment, the invention discloses a method of simultaneously amplifying target sequences from Salmonella spp. and E. coli O157: H7 in a sample in the presence of a primer pairs specific to Salmonella spp., primer pairs specific to E. coli O157:H7, and a nucleic acid polymerase, wherein a concentration of the nucleic acid polymerase is equal to or higher than 0.1 unit/μl. The amplification includes hybridizing the primers specific to Salmonella spp. and E. coli O157: H7 target sequences, and extending a primer of the hybridization product by a nucleic acid polymerase that is template-dependent, thereby producing an extended primer product. The amplifying is performed by using, for example, an amplification method selected from the group consisting of polymerase chain reaction (PCR), rolling circle amplification (RCA), nucleic acid sequence based amplification (NASBA), and strand displacement amplification (SDA). The hybridization means formation of a duplex by complementarily linking strands of a 2-stranded nucleic acid. The hybridization may be performed by using any known method in the art. For example, the hybridization may be performed by separating a duplex into single strands by heating a primer and/or a target sequence and cooling to allow two complementary strands to be linked. If the target sequence is a single strand, the separation of the primer and/or the target sequence may not be needed. The hybridization may be performed using a buffer that is appropriate for the kind of the selected primer and/or target sequence selected, for example, a buffer with an appropriate salt concentration and an appropriate pH. The extension is well known in the art. The extension may be performed by using, for example, a DNA polymerase, a RNA polymerase, or a reverse transcriptase. The nucleic acid polymerase may be thermally stable, for example, may retain its activity when exposed to a temperature of 95° C. or more. A thermostable DNA polymerase may be an enzyme separated from thermophilic bacteria as defined herein. For example, the thermally stable DNA polymerase may be a Taq polymerase having an optimal activity at a temperature of about 70° C.

The number of copies of the target sequence from Salmonella spp. may be ten or more times greater than the number of copies of the target sequence from E. coli O157: H7 in the sample, or vice versa. For example, a ratio of the number of copies of the target sequence from Salmonella spp. and the number of copies of the target sequence from E. coli O157: H7 in the sample is equal to or greater than 10:1, or equal to or smaller than 1:10.

### Reverse Transcriptase-PCR Amplification of a RNA Target Nucleic Acid Sequence

One of the most widely used techniques to study gene expression exploits first-strand cDNA for mRNA sequence(s) as template for amplification by the PCR.

The term “reverse transcriptase activity” and “reverse transcription” refers to the enzymatic activity of a class of polymerases characterized as RNA-dependent DNA polymerases that can synthesize a DNA strand (i.e., complementary DNA, cDNA) utilizing an RNA strand as a template.

“Reverse transcriptase-PCR” of “RNA PCR” is a PCR reaction that uses RNA template and a reverse transcriptase, or an enzyme having reverse transcriptase activity, to first generate a single stranded DNA molecule prior to the multiple cycles of DNA-dependent DNA polymerase primer elongation. Multiplex PCR refers to PCR reactions that produce more than one amplified product in a single reaction, typically by the inclusion of more than two primers in a single reaction.

Exemplary reverse transcriptases include, but are not limited to, the Moloney murine leukemia virus (M-MLV) RT as described in U.S. Pat. No. 4,943,531, a mutant form of M-MLV-RT lacking RNase H activity as described in U.S. Pat. No. 5,405,776, bovine leukemia virus (BLV) RT, Rous sarcoma virus (RSV) RT, Avian Myeloblastosis Virus (AMV) RT and reverse transcriptases disclosed in U.S. Pat. No. 7,883,871.

The reverse transcriptase-PCR procedure, carried out as either an end-point or real-time assay, involves two separate molecular syntheses: (i) the synthesis of cDNA from an RNA template; and (ii) the replication of the newly synthesized cDNA through PCR amplification. To attempt to address the technical problems often associated with reverse transcriptase-PCR, a number of protocols have been developed taking into account the three basic steps of the procedure: (a) the denaturation of RNA and the hybridization of reverse primer; (b) the synthesis of cDNA; and (c) PCR amplification. In the so called “uncoupled” reverse transcriptase-PCR procedure (e.g., two step reverse transcriptase-PCR), reverse transcription is performed as an independent step using the optimal buffer condition for reverse transcriptase activity. Following cDNA synthesis, the reaction is diluted to decrease MgCl₂, and deoxyribonucleoside triphosphate (dNTP) concentrations to conditions optimal for Taq DNA Polymerase activity, and PCR is carried out according to standard conditions (see U.S. Pat. Nos. 4,683,195 and 4,683,202). By contrast, “coupled” RT PCR methods use a common or compromised buffer for reverse transcriptase and Taq DNA Polymerase activities. In one version, the annealing of reverse primer is a separate step preceding the addition of enzymes, which are then added to the single reaction vessel. In another version, the reverse transcriptase activity is a component of the thermostable Tth DNA polymerase. Annealing and cDNA synthesis are performed in the presence of Mn²⁺ then PCR is carried out in the presence of Mg²⁺ after the removal of Mn²⁺ by a chelating agent. Finally, the “continuous” method (e.g., one step reverse transcriptase-PCR) integrates the three reverse transcriptase-PCR steps into a single continuous reaction that avoids the opening of the reaction tube for component or enzyme addition. Continuous reverse transcriptase-PCR has been described as a single enzyme system using the reverse transcriptase activity of thermostable Taq DNA Polymerase and Tth polymerase and as a two enzyme system using AMV RT and Taq DNA Polymerase wherein the initial 65° C. RNA denaturation step may be omitted.

One step reverse transcriptase-PCR provides several advantages over uncoupled reverse transcriptase-PCR. One step reverse transcriptase-PCR requires less handling of the reaction mixture reagents and nucleic acid products than uncoupled reverse transcriptase-PCR (e.g., opening of the reaction tube for component or enzyme addition in between the two reaction steps), and is therefore less labor intensive, reducing the required number of person hours. One step reverse transcriptase-PCR also requires less sample, and reduces the risk of contamination. The sensitivity and specificity of one-step reverse transcriptase-PCR has proven well suited for studying expression levels of one to several genes in a given sample or the detection of pathogen RNA. Typically, this procedure has been limited to use of gene-specific primers to initiate cDNA synthesis.

The ability to measure the kinetics of a PCR reaction by on-line detection in combination with these reverse transcriptase-PCR techniques has enabled accurate and precise measurement of RNA sequences with high sensitivity. This has become possible by detecting the reverse transcriptase-PCR product through fluorescence monitoring and measurement of PCR product during the amplification process by fluorescent dual-labeled hybridization probe technologies, such as the 5′ fluorogenic nuclease assay (“TaqMan™”) or endonuclease assay (“CataCleave™”), discussed below.

### Real-time PCR Using a CataCleave™ Probe

Post-amplification amplicon detection can be both laborious and time consuming. Real-time methods have been developed to monitor amplification during the PCR process. These methods typically employ fluorescently labeled probes that bind to the newly synthesized DNA or dyes whose fluorescence emission is increased when intercalated into double stranded DNA.

The probes are generally designed so that donor emission is quenched in the absence of target by fluorescence resonance energy transfer (FRET) between two chromophores. The donor chromophore, in its excited state, may transfer energy to an acceptor chromophore when the pair is in close proximity. This transfer is always non-radiative and occurs through dipole-dipole coupling. Any process that sufficiently increases the distance between the chromophores will decrease FRET efficiency such that the donor chromophore emission can be detected radiatively. Common donor chromophores include FAM, TAMRA, VIC, JOE, Cy3, Cy5, and Texas Red.) Acceptor chromophores are chosen so that their excitation spectra overlap with the emission spectrum of the donor. An example of such a pair is FAM-TAMRA. There are also non fluorescent acceptors that will quench a wide range of donors. Other examples of appropriate donor-acceptor FRET pairs will be known to those skilled in the art.

Common examples of FRET probes that can be used for real-time detection of PCR include molecular beacons(e.g., U.S. Pat. No. 5,925,517), TaqMan™ probes (e.g., U.S. Pat. Nos. 5,210,015 and 5,487,972), and CataCleave™ probes (e.g., U.S. Pat. No. 5,763,181). The molecular beacon is a single stranded oligonucleotide designed so that in the unbound state the probe forms a secondary structure where the donor and acceptor chromophores are in close proximity and donor emission is reduced. At the proper reaction temperature the beacon unfolds and specifically binds to the amplicon. Once unfolded the distance between the donor and acceptor chromophores increases such that FRET is reversed and donor emission can be monitored using specialized instrumentation. TaqMan™ and CataCleave™ technologies differ from the molecular beacon in that the FRET probes employed are cleaved such that the donor and acceptor chromophores become sufficiently separated to reverse FRET.

TaqMan™ technology employs a single stranded oligonucleotide probe that is labeled at the 5′ end with a donor chromophore and at the 3′ end with an acceptor chromophore. The DNA polymerase used for amplification must contain a 5′->3′ exonuclease activity. The TaqMan™ probe binds to one strand of the amplicon at the same time that the primer binds. As the DNA polymerase extends the primer the polymerase will eventually encounter the bound TaqMan™ probe. At this time the exonuclease activity of the polymerase will sequentially degrade the TaqMan™ probe starting at the 5′ end. As the probe is digested the mononucleotides comprising the probe are released into the reaction buffer. The donor diffuses away from the acceptor and FRET is reversed. Emission from the donor is monitored to identify probe cleavage. Because of the way TaqMan works a specific amplicon can be detected only once for every cycle of PCR. Extension of the primer through the TaqMan™ target site generates a double stranded product that prevents further binding of TaqMan™ probes until the amplicon is denatured in the next PCR cycle.

U.S. Pat. No. 5,763,181, of which content is incorporated herein by reference, describes another real-time detection method (referred to as “CataCleave™”). CataCleave™ technology differs from TaqMan™ in that cleavage of the probe is accomplished by a second enzyme that does not have polymerase activity. The CataCleave™ probe has a sequence within the molecule which is a target of an endonuclease, such as, for example a restriction enzyme or RNAase. In one example, the CataCleave™ probe has a chimeric structure where the 5′ and 3′ ends of the probe are constructed of DNA and the cleavage site contains RNA.

For example, the probe may have a structure represented by Formula I below:

R1-X-R2 (Formula I)

wherein R1 and R2 are each selected from the group consisting of a nucleic acid and a nucleic acid analog, and X may be a first RNA. For example, R1 and R2 may all be DNA; R1 may be DNA and R2 may be RNA; R1 may be RNA and R2 may be DNA; or R1 and R2 may all be RNA. The nucleic acid or nucleic acid analog of R1 and R2 may be a protected nucleic acid. For example, the nucleic acid and the nucleic acid analog may be methylated and thus, may be resistant to decomposition due to an RNA specific decomposition enzyme (for example, RNase H). A length of the probe may differ according to a target sequence and a PCR condition. An annealing temperature (Tm) of the probe may be about 60° C. or more, about 70° C. or more, or about 80° C. or more.

The probe may be modified. For example, in the probe, a base may be partially or entirely methylated. Such modification of a base may protect the probe from decomposition by an enzyme, a chemical factor, or other factors. In addition, in the probe, —OH at a 5′ end or 3′ end may be blocked. For example, —OH at the 3′ end of the probe may be blocked and thus, the probe may not be a substrate for primer extension by the template-dependent nucleic acid polymerase.

The DNA sequence portions of the probe can be labeled with a FRET pair either at the ends or internally. The PCR reaction includes an RNase H enzyme that will specifically cleave the RNA sequence portion of a RNA-DNA duplex. After cleavage, the two halves of the probe dissociate from the target amplicon at the reaction temperature and diffuse into the reaction buffer. As the donor and acceptors separate FRET is reversed in the same way as the TaqMan™ probe and donor emission can be monitored. Cleavage and dissociation regenerates a site for further CataCleave™ binding. In this way it is possible for a single amplicon to serve as a target or multiple rounds of probe cleavage until the primer is extended through the CataCleave™ probe binding site.

### Labeling of a CataCleave Probe

The term “probe” comprises a polynucleotide that comprises a specific portion designed to hybridize in a sequence-specific manner with a complementary region of a specific nucleic acid sequence, e.g., a target nucleic acid sequence. A length of the probe may be in the range of, for example, about 10 to about 200 nucleotides, about 15 to about 200 nucleotides, or about 15 to about 60 nucleotides in length, more preferably, about 18 to about 30 nucleotides in length. The precise sequence and length of an oligonucleotide probe of the invention depends in part on the nature of the target polynucleotide to which it binds. The binding location and length may be varied to achieve appropriate annealing and melting properties for a particular embodiment. Guidance for making such design choices can be found in many of the references describing TaqMan™ assays or CataCleave™, described in U.S. Pat. Nos. 5,763,181, 6,787,304, and 7,112,422, of which contents are incorporated herein by reference.

In certain embodiments, the probe is “substantially complementary” to the target nucleic acid sequence.

As used herein, the term “substantially complementary” refers to two nucleic acid strands that are sufficiently complimentary in sequence to anneal and form a stable duplex. The complementarity does not need to be perfect; there may be any number of base pair mismatches, for example, between the two nucleic acids. However, if the number of mismatches is so great that no hybridization can occur under even the least stringent hybridization conditions, the sequence is not a substantially complementary sequence. When two sequences are referred to as “substantially complementary” herein, it means that the sequences are sufficiently complementary to each other to hybridize under the selected reaction conditions. The relationship of nucleic acid complementarity and stringency of hybridization sufficient to achieve specificity is well known in the art. Two substantially complementary strands can be, for example, perfectly complementary or can contain from 1 to many mismatches so long as the hybridization conditions are sufficient to allow, for example discrimination between a pairing sequence and a non-pairing sequence. Accordingly, “substantially complementary” sequences can refer to sequences with base-pair complementarity of 100, 95, 90, 80, 75, 70, 60, 50 percent or less, or any number in between, in a double-stranded region.

As used herein, “label” or “detectable label” of the CataCleave probe refers to any moiety that is detectable by using a spectroscopic, photo-chemical, biochemical, immunochemical, or chemical method. The detectable label may be selected from the group consisting of an enzyme, an enzyme substrate, a radioactive material, a fluorescent dye, a chromophore, a chemi-luminescence label, an electrochemical luminescence label, a ligand having a particular bonding partner, and other labels that interact with each other to increase, change, or reduce a signal. The detectable label may survive during heat cycling of a PCR.

The detectable label may be a fluorescence resonance energy transfer (FRET) pair. The detectable label may be a FRET pair, and a fluorescence donor and a fluorescence receptor may be spaced apart from each other at an appropriate interval and thus, fluorescence donor emission is hindered and is activated by disassociation caused by cleaving. That is, in the probe, when the probe is not cleaved, a fluorescence donor emission is quenched by a fluorescence acceptor emission by FRET between two chromophores. When a donor chromophore is located near the acceptor chromophore, a donor chromophore in an excited state may transfer energy to an acceptor chromophore. The transfer is always non-radiative and may occur by dipole-dipole coupling. If the distance between two chromophores is sufficiently increased, FRET efficiency is decreased and the donor chromophore emission may be radiatively detected.

In one embodiment, the detectable label can be a fluorochrome compound that is attached to the probe by covalent or non-covalent means.

As used herein, “fluorochrome” refers to a fluorescent compound that emits light upon excitation by light of a shorter wavelength than the light that is emitted. The term “fluorescent donor” or “fluorescence donor” refers to a fluorochrome that emits light that is measured in the assays described in the present invention. More specifically, a fluorescent donor provides light that is absorbed by a fluorescence acceptor. The term “fluorescent acceptor” or “fluorescence acceptor” refers to either a second fluorochrome or a quenching molecule that absorbs light emitted from the fluorescence donor. The second fluorochrome absorbs the light that is emitted from the fluorescence donor and emits light of longer wavelength than the light emitted by the fluorescence donor. The quenching molecule absorbs light emitted by the fluorescence donor.

Any luminescent molecule, preferably a fluorochrome and/or fluorescent quencher may be used in the practice of this invention, including, for example, Alexa Fluor™ 350, Alexa Fluor™ 430, Alexa Fluor™ 488, Alexa Fluor™ 532, Alexa Fluor™ 546, Alexa Fluor™ 568, Alexa Fluor™ 594, Alexa Fluor™ 633, Alexa Fluor™ 647, Alexa Fluor™ 660, Alexa Fluor™ 680, 7-diethylaminocoumarin-3-carboxylic acid, Fluorescein, Oregon Green 488, Oregon Green 514, Tetramethylrhodamine, Rhodamine X, Texas Red dye, QSY 7, QSY33, Dabcyl, BODIPY FL, BODIPY 630/650, BODIPY 6501665, BODIPY TMR-X, BODIPY TR-X, Dialkylaminocoumarin, Cy5.5, Cy5, Cy3.5, Cy3, DTPA(Eu³⁺)-AMCA and TTHA(Eu³⁺)AMCA.

In one embodiment, the 3′ terminal nucleotide of the oligonucleotide probe is blocked or rendered incapable of extension by a nucleic acid polymerase. Such blocking is conveniently carried out by the attachment of a reporter or quencher molecule to the terminal 3′ position of the probe.

In one embodiment, reporter molecules are fluorescent organic dyes derivatized for attachment to the terminal 3′ or terminal 5′ ends of the probe via a linking moiety. Preferably, quencher molecules are also organic dyes, which may or may not be fluorescent, depending on the embodiment of the invention. For example, in a preferred embodiment of the invention, the quencher molecule is fluorescent. Generally whether the quencher molecule is fluorescent or simply releases the transferred energy from the reporter by non-radiative decay, the absorption band of the quencher should substantially overlap the fluorescent emission band of the reporter molecule. Non-fluorescent quencher molecules that absorb energy from excited reporter molecules, but which do not release the energy radiatively, are referred to in the application as chromogenic molecules.

Exemplary reporter-quencher pairs may be selected from xanthene dyes, including fluoresceins, and rhodamine dyes. Many suitable forms of these compounds are widely available commercially with substituents on their phenyl moieties which can be used as the site for bonding or as the bonding functionality for attachment to an oligonucleotide. Another group of fluorescent compounds are the naphthylamines, having an amino group in the alpha or beta position. Included among such naphthylamino compounds are 1-dimethylaminonaphthyl-5-sulfonate, 1-anilino-8-naphthalene sulfonate and 2-p-touidinyl6-naphthalene sulfonate. Other dyes include 3-phenyl-7-isocyanatocoumarin, acridines, such as 9-isothiocyanatoacridine and acridine orange, N-(p-(2-benzoxazolyl)phenyl)maleimide, benzoxadiazoles, stilbenes, pyrenes, and the like.

In one embodiment, reporter and quencher molecules are selected from fluorescein and rhodamine dyes.

There are many linking moieties and methodologies for attaching reporter or quencher molecules to the 5′ or 3′ termini of oligonucleotides, as exemplified by the following references: Eckstein, editor, Oligonucleotides and Analogues: A Practical Approach (IRL Press, Oxford, 1991); Zuckerman et al., Nucleic Acids Research, 15: 5305-5321 (1987) (3′ thiol group on oligonucleotide); Sharma et al., Nucleic Acids Research, 19: 3019 (1991) (3′ sulfhydryl); Giusti et al., PCR Methods and Applications, 2: 223-227 (1993) and Fung et al., U.S. Pat. No. 4,757,141 (5′ phosphoamino group via Aminolink™ II available from Applied Biosystems, Foster City, Calif.) Stabinsky, U.S. Pat. No. 4,739,044 (3′ aminoalkylphosphoryl group); Agrawal et al., Tetrahedron Letters, 31: 1543-1546 (1990) (attachment via phosphoramidate linkages); Sproat et al., Nucleic Acids Research, 15: 4837 (1987) (5′ mercapto group); Nelson et al., Nucleic Acids Research, 17: 7187-7194 (1989) (3′ amino group); and the like.

Rhodamine and fluorescein dyes are also conveniently attached to the 5′ hydroxyl of an oligonucleotide at the conclusion of solid phase synthesis by way of dyes derivatized with a phosphoramidite moiety, e.g., Woo et al., U.S. Pat. No. 5,231,191; and Hobbs, Jr., U.S. Pat. No. 4,997,928.

### Attachment of a CataCleave Probe to a Solid Support

In one embodiment, the oligonucleotide probe can be attached to a solid support. Different probes may be attached to the solid support and may be used to simultaneously detect different target sequences in a sample. Reporter molecules having different fluorescence wavelengths can be used on the different probes, thus enabling hybridization to the different probes to be separately detected.

Examples of preferred types of solid supports for immobilization of the oligonucleotide probe include controlled pore glass, glass plates, polystyrene, avidin coated polystyrene beads cellulose, nylon, acrylamide gel and activated dextran, controlled pore glass (CPG), glass plates and high cross-linked polystyrene. These solid supports are preferred for hybridization and diagnostic studies because of their chemical stability, ease of functionalization and well defined surface area. Solid supports such as controlled pore glass (500 Å, 1000 Å) and non-swelling high cross-linked polystyrene (1000 Å) are particularly preferred in view of their compatibility with oligonucleotide synthesis.

The oligonucleotide probe may be attached to the solid support in a variety of manners. For example, the probe may be attached to the solid support by attachment of the 3′ or 5′ terminal nucleotide of the probe to the solid support. However, the probe may be attached to the solid support by a linker which serves to distance the probe from the solid support. The linker is most preferably at least 30 atoms in length, more preferably at least 50 atoms in length.

Hybridization of a probe immobilized to a solid support generally requires that the probe be separated from the solid support by at least 30 atoms, more-preferably at least 50 atoms. In order to achieve this separation, the linker generally includes a spacer positioned between the linker and the 3′ nucleoside. For oligonucleotide synthesis, the linker arm is usually attached to the 3′-OH of the 3′ nucleoside by an ester linkage which can be cleaved with basic reagents to free the oligonucleotide from the solid support.

A wide variety of linkers are known in the art which may be used to attach the oligonucleotide probe to the solid support. The linker may be formed of any compound which does not significantly interfere with the hybridization of the target sequence to the probe attached to the solid support. The linker may be formed of a homopolymeric oligonucleotide which can be readily added on to the linker by automated synthesis. Alternatively, polymers such as functionalized polyethylene glycol can be used as the linker. Such polymers are preferred over homopolymeric oligonucleotides because they do not significantly interfere with the hybridization of probe to the target oligonucleotide. Polyethylene glycol is particularly preferred because it is commercially available, soluble in both organic and aqueous media, easy to functionalize, and completely stable under oligonucleotide synthesis and post-synthesis conditions.

The linkages between the solid support, the linker and the probe are preferably not cleaved during removal of base protecting groups under basic conditions at high temperature. Examples of preferred linkages include carbamate and amide linkages. Immobilization of a probe is well known in the art and one skilled in the art may determine the immobilization conditions.

According to one embodiment of the method, the CataCleave probe is immobilized on a solid support. The oligonucleotide probe is contacted with a sample of nucleic acids under conditions favorable for hybridization between target sequence in a sample and CataCleave probe. The fluorescence signal of the reporter molecule is measured before and after being contacted with the sample. Since the reporter molecule on the probe exhibits a greater fluorescence signal when the probe is hybridized to a target sequence, an increase in the fluorescence signal after the probe is contacted with the sample indicates the hybridization of the probe to target sequences in the sample. Immobilization of the probe to the solid support enables the target sequence hybridized to the probe to be readily isolated from the sample. In later steps, the isolated target sequence may be separated from the solid support and processed (e.g., purified, amplified) according to methods well known in the art depending on the particular needs of the researcher.

### RNase H Cleavage of the CataCleave™ Probe

RNase H hydrolyzes RNA in RNA-DNA hybrids. First identified in calf thymus, RNase H has subsequently been described in a variety of organisms. Indeed, RNase H activity appears to be ubiquitous in eukaryotes and bacteria. Although RNase Hs form a family of proteins of varying molecular weight and nucleolytic activity, substrate requirements appear to be similar for the various isotypes. For example, most RNase Hs studied to date function as endonucleases and require divalent cations (e.g., Mg²⁺, Mn²⁺) to produce cleavage products with 5′ phosphate and 3′ hydroxyl termini.

In prokaryotes, RNase H have been cloned and extensively characterized (see Crooke, et al., (1995) Biochem J, 312 (Pt 2), 599-608; Lima, et al., (1997) J Biol Chem, 272, 27513-27516; Lima, et al., (1997) Biochemistry, 36, 390-398; Lima, et al., (1997) J Biol Chem, 272, 18191-18199; Lima, et al., (2007) Mol Pharmacol, 71, 83-91; Lima, et al., (2007) Mol Pharmacol, 71, 73-82; Lima, et al., (2003) J Biol Chem, 278, 14906-14912; Lima, et al., (2003) J Biol Chem, 278, 49860-49867; Itaya, M., Proc. Natl. Acad. Sci. USA, 1990, 87, 8587-8591). For example, E. coli RNase HII is 213 amino acids in length whereas RNase HI is 155 amino acids long. E. coli RNase HII displays only 17% homology with E. coli RNase HI. An RNase H cloned from S. typhimurium differed from E. coli RNase HI in only 11 positions and was 155 amino acids in length (Itaya, M. and Kondo K., Nucleic Acids Res., 1991, 19, 4443-4449).

Proteins that display RNase H activity have also been cloned and purified from a number of viruses, other bacteria and yeast (Wintersberger, U. Pharmac. Ther., 1990, 48, 259-280). In many cases, proteins with RNase H activity appear to be fusion proteins in which RNase H is fused to the amino or carboxy end of another enzyme, often a DNA or RNA polymerase. The RNase H domain has been consistently found to be highly homologous to E. coli RNase HI, but because the other domains vary substantially, the molecular weights and other characteristics of the fusion proteins vary widely.

In higher eukaryotes two classes of RNase H have been defined based on differences in molecular weight, effects of divalent cations, sensitivity to sulfhydryl agents and immunological cross-reactivity (Busen et al., Eur. J. Biochem., 1977, 74, 203-208). RNase HI enzymes are reported to have molecular weights in the 68-90 kDa range, be activated by either Mn²⁺ or Mg²⁺ and be insensitive to sulfhydryl agents. In contrast, RNase H II enzymes have been reported to have molecular weights ranging from 31-45 kDa, to require Mg²⁺ to be highly sensitive to sulfhydryl agents and to be inhibited by Mn²⁺ (Busen, W., and Hausen, P., Eur. J. Biochem., 1975, 52, 179-190; Kane, C. M., Biochemistry, 1988, 27, 3187-3196; Busen, W., J. Biol. Chem., 1982, 257, 7106-7108).

An enzyme with RNase HII characteristics has also been purified to near homogeneity from human placenta (Frank et al., Nucleic Acids Res., 1994, 22, 5247-5254). This protein has a molecular weight of approximately 33 kDa and is active in a pH range of 6.5-10, with a pH optimum of 8.5-9. The enzyme requires Mg²⁺ and is inhibited by Mn²⁺ and n-ethyl maleimide. The products of cleavage reactions have 3′ hydroxyl and 5′ phosphate termini.

A detailed comparison of RNases from different species is reported in Ohtani N, Haruki M, Morikawa M, Kanaya S. J Biosci Bioeng. 1999; 88(1):12-9.

Examples of RNase H enzymes, which may be employed in the embodiments, also include, but are not limited to, thermostable RNase H enzymes isolated from thermophilic organisms such as Pyrococcus furiosus RNase HII, Pyrococcus horikoshi RNase HII, Thermococcus litoralis RNase HI, Thermus thermophilus RNase HI.

Other RNase H enzymes that may be employed in the embodiments are described in, for example, U.S. Pat. No. 7,422,888 to Uemori or the published U.S. Patent Application No. 2009/0325169 to Walder, the contents of which are incorporated herein by reference.

In one embodiment, an RNase H enzyme is a thermostable RNase H with 40%, 50%, 60%, 70%, 80%, 90%, 95% or 99% homology with the amino acid sequence of Pfu RNase H11 (SEQ ID NO: 8), shown below.

| (SEQ ID NO: 8) | |
|---|---|
| MKIGGIDEAG RGPAIGPLVV ATVVVDEKNI EKLRNIGVKD SKQLTPHERK NLFSQITSIA | 60 |
| DDYKIVIVSP EEIDNRSGTM NELEVEKFAL ALNSLQIKPA LIYADAADVD ANRFASLIER | 120 |
| RLNYKAKIIA EHKADAKYPV VSAASILAKV VRDEEIEKLK KQYGDFGSGY PSDPKTKKWL | 180 |
| EEYYKKHNSF PPIVRRTWET VRKIEESIKA KKSQLTLDKF FKKP | |

The homology can be determined using, for example, a computer program DNASIS-Mac (Takara Shuzo), a computer algorithm FASTA (version 3.0; Pearson, W. R. et al., Pro. Natl. Acad. Sci., 85:2444-2448, 1988) or a computer algorithm BLAST (version 2.0, Altschul et al., Nucleic Acids Res. 25:3389-3402, 1997)

In another embodiment, an RNase H enzyme is a thermostable RNase H with at least one or more homology regions 1-4 corresponding to positions 5-20, 33-44, 132-150, and 158-173 of SEQ ID NO: 8.

HOMOLOGY REGION 1: GIDEAG RGPAIGPLVV (SEQ ID NO:
9; corresponding to positions 5-20 of SEQ ID NO:
8)
HOMOLOGY REGION 2: LRNIGVKD SKQL (SEQ ID NO: 10;
corresponding to positions 33-44 of SEQ ID NO: 8)
HOMOLOGY REGION 3: HKADAKYPV VSAASILAKV (SEQ ID
NO: 11; corresponding to positions 132-150 of SEQ
ID NO: 8)
HOMOLOGY REGION 4: KLK KQYGDFGSGY PSD (SEQ ID NO:
12; corresponding to positions 158-173 of SEQ ID
NO: 8)

In another embodiment, an RNase H enzyme is a thermostable RNase H with at least one of the homology regions having 50%, 60%. 70%, 80%, 90% sequence identity with a polypeptide sequence of SEQ ID NOs: 9, 10, 11 or 12.

The terms “sequence identity” as used herein refers to the extent that sequences are identical or functionally or structurally similar on a amino acid to amino acid basis over a window of comparison. Thus, a “percentage of sequence identity”, for example, can be calculated by comparing two optimally aligned sequences over the window of comparison, determining the number of positions at which the identical amino acid occurs in both sequences to yield the number of matched positions, dividing the number of matched positions by the total number of positions in the window of comparison (i.e., the window size), and multiplying the result by 100 to yield the percentage of sequence identity.

In certain embodiments, the RNase H can be modified to produce a hot start “inducible” RNase H.

The term “modified RNase H,” as used herein, can be an RNase H reversely coupled to or reversely bound to an inhibiting factor that causes the loss of the endonuclease activity of the RNase H. Release or decoupling of the inhibiting factor from the RNase H restores at least partial or full activity of the endonuclease activity of the RNase H. About 30-100% of its activity of an intact RNase H may be sufficient. The inhibiting factor may be a ligand or a chemical modification. The ligand can be an antibody, an aptamer, a receptor, a cofactor, or a chelating agent. The ligand can bind to the active site of the RNase H enzyme thereby inhibiting enzymatic activity or it can bind to a site remote from the RNase's active site. In some embodiment, the ligand may induce a conformational change. The chemical modification can be a crosslinking (for example, by formaldehyde) or acylation. The release or decoupling of the inhibiting factor from the RNase HII may be accomplished by heating a sample or a mixture containing the coupled RNase HII (inactive) to a temperature of about 65° C. to about 95° C. or higher, and/or lowering the pH of the mixture or sample to about 7.0 or lower.

As used herein, a hot start “inducible” RNase H activity refers to the herein described modified RNase H that has an endonuclease catalytic activity that can be regulated by association with a ligand. Under permissive conditions, the RNase H endonuclease catalytic activity is activated whereas at non-permissive conditions, this catalytic activity is inhibited. In some embodiments, the catalytic activity of a modified RNase H can be inhibited at temperature conducive for reverse transcription, i.e. about 42° C., and activated at more elevated temperatures found in PCR reactions, i.e. about 65° C. to 95° C. A modified RNase H with these characteristics is said to be “heat inducible.”

In other embodiments, the catalytic activity of a modified RNase H can be regulated by changing the pH of a solution containing the enzyme.

As used herein, a “hot start” enzyme composition refers to compositions having an enzymatic activity that is inhibited at non-permissive temperatures, i.e. from about 25° C. to about 45° C. and activated at temperatures compatible with a PCR reaction, e.g. about 55° C. to about 95° C. In certain embodiment, a “hot start” enzyme composition may have a ‘hot start’ RNase H and/or a ‘hot start’ thermostable DNA polymerase that are known in the art.

Crosslinking of RNase H enzymes can be performed using, for example, formaldehyde. In one embodiment, a thermostable RNase HII is subjected to controlled and limited crosslinking using formaldehyde. By heating an amplification reaction composition, which comprises the modified RNase HII in an active state, to a temperature of about 95° C. or higher for an extended time, for example about 15 minutes, the crosslinking is reversed and the RNase HII activity is restored.

In general, the lower the degree of crosslinking, the higher the endonuclease activity of the enzyme is after reversal of crosslinking. The degree of crosslinking may be controlled by varying the concentration of formaldehyde and the duration of crosslinking reaction. For example, about 0.2% (w/v), about 0.4% (w/v), about 0.6% (w/v), or about 0.8% (w/v) of formaldehyde may be used to crosslink an RNase H enzyme. About 10 minutes of crosslinking reaction using 0.6% formaldehyde may be sufficient to inactivate RNase HII from Pyrococcus furiosus.

The crosslinked RNase HII does not show any measurable endonuclease activity at about 37° C. In some cases, a measurable partial reactivation of the crosslinked RNase HII may occur at a temperature of around 50° C., which is lower than the PCR denaturation temperature. To avoid such unintended reactivation of the enzyme, it may be required to store or keep the modified RNase HII at a temperature lower than 50° C. until its reactivation.

In general, PCR requires heating the amplification composition at each cycle to about 95° C. to denature the double stranded target sequence which will also release the inactivating factor from the RNase H, partially or fully restoring the activity of the enzyme.

RNase H may also be modified by subjecting the enzyme to acylation of lysine residues using an acylating agent, for example, a dicarboxylic acid. Acylation of RNase H may be performed by adding cis-aconitic anhydride to a solution of RNase H in an acylation buffer and incubating the resulting mixture at about 1-20° C. for 5-30 hours. In one embodiment, the acylation may be conducted at around 3-8° C. for 18-24 hours. The type of the acylation buffer is not particularly limited. In an embodiment, the acylation buffer has a pH of between about 7.5 to about 9.0.

The activity of acylated RNase H can be restored by lowering the pH of the amplification composition to about 7.0 or less. For example, when Tris buffer is used as a buffering agent, the composition may be heated to about 95° C., resulting in the lowering of pH from about 8.7 (at 25° C.) to about 6.5 (at 95° C.).

The duration of the heating step in the amplification reaction composition may vary depending on the modified RNase H, the buffer used in the PCR, and the like. However, in general, heating the amplification composition to 95° C. for about 30 seconds-4 minutes is sufficient to restore RNase H activity. In one embodiment, using a commercially available buffer such as Invitrogen AgPath™ buffer (a Tris based buffer (pH 7.6) and one or more non-ionic detergents, full activity of Pyrococcus furiosus RNase HII is restored after about 2 minutes of heating.

RNase H activity may be determined using methods that are well in the art. For example, according to a first method, the unit activity is defined in terms of the acid-solubilization of a certain number of moles of radiolabeled polyadenylic acid in the presence of equimolar polythymidylic acid under defined assay conditions (see Epicentre Hybridase thermostable RNase HI). In the second method, unit activity is defined in terms of a specific increase in the relative fluorescence intensity of a reaction containing equimolar amounts of the probe and a complementary template DNA under defined assay conditions.

### Real-Time Detection of Salmonella and E. Coli O157: H7 Nucleic Acid Sequences

The labeled Salmonella-specific and E. coli O157: H7-specific oligonucleotide probes may be used for the real-time detection of Salmonella and E. coli O157: H7 in a sample.

A CataCleave™ oligonucleotide probe is first synthesized with DNA and RNA sequences that are complimentary to Salmonella and E. coli O157: H7 target sequences. The probe can be labeled, for example, with a FRET pair, for example, a fluorescein molecule at one end of the probe and a rhodamine quencher molecule at the other end. The probe can be synthesized to be substantially complementary to a target nucleic acid sequence.

In one embodiment, real-time nucleic acid amplification is performed on nucleic acids extracted from a sample or in a cell lysate in the presence of a thermostable nucleic acid polymerase, a RNase H activity, PCR amplification primers pairs capable of hybridizing to Salmonella and E. coli O157: H7 target sequences, and labeled Salmonella and E. coli O157: H7 CataCleave™ oligonucleotide probes.

In one embodiment, the simultaneous amplification method may include a PCR reaction mixture containing Salmonella F1 primer (e.g., SEQ ID NO: 1) and Sal-InvR2 primer (e.g., SEQ ID NO: 2), O157 I-F1 primer (e.g., SEQ ID NO: 3) and O157 I-R primer (e.g., SEQ ID NO:4), inv-CC-probe2 (FAM) (e.g., SEQ ID NO: 5), O157 I-P2 probe(CY3) (e.g., SEQ ID NO: 6), RNase H11, and Taq polymerase. Thus, Salmonella and E. coli O157: H7 in a sample may be simultaneously and efficiently amplified and detected. The reaction mixture may optionally further contain Salmonella and E. coli O157: H7 internal amplification control probes, of which example for Salmonella is shown as SEQ ID NO: 7.

During the real-time PCR reaction, RNase H cleavage of the RNA: DNA heteroduplex probe formed between the RNA moiety of a CataCleave™ oligonucleotide probe and the Salmonella and E. coli O157: H7-specific target sequences in the PCR amplicons leads to the separation of the fluorescent donor from the fluorescent quencher and results in the real-time increase in fluorescence of the probe corresponding to the real-time detection of the Salmonella and E. coli O157: H7 target sequences in the sample.

### Kits

The disclosure herein also provides for a kit format which comprises a package unit having one or more reagents for the real-time detection of Salmonella and E. coli O157: H7 target sequences in a sample. The kit may also contain one or more of the following items: buffers, instructions, and positive or negative controls. Kits may include containers of reagents mixed together in suitable proportions for performing the methods described herein. Reagent containers preferably contain reagents in unit quantities that obviate measuring steps when performing the subject methods.

Kits may also contain reagents for real-time PCR including, but not limited to, a thermostable polymerase, RNase H, Salmonella and E. coli O157: H7 specific primers and Salmonella and E. coli O157: H7 labeled CataCleave™ oligonucleotide probes that can anneal to the real-time Salmonella and E. coli O157: H7 PCR products and allow for the detection of the Salmonella and E. coli O157: H7 target nucleic acid sequences according to the methodology described herein. In another embodiment, the kit reagents further comprise reagents for the extraction of total genomic DNA, total RNA or polyA⁺ RNA from a sample. Kit reagents may also include reagents for reverse transcriptase-PCR analysis where applicable.

In one embodiment, a kit for simultaneously amplifying and detecting target sequences from Salmonella spp. and E. coli O157: H7 in a sample, includes a Salmonella F1 primer, a Sal-InvR2 primer, an O157 I-F1 primer, an O157 I-R primer, an inv-CC-probe2 (FAM), an E. coli O157 I-P2 probe (CY3), a RNase HII, and a nucleic acid polymerase.

The nucleic acid polymerase may be Taq polymerase. The nucleic acid polymerase may have a concentration of 0.1 unit/μL or more. The nucleic acid polymerase with a concentration of 0.1 unit/μL or more may be included in a reaction mixture. For example, the nucleic acid polymerase with a concentration of 0.1 to 10 unit/μL, 0.1 to 5 unit/μL, 0.1 to 2.5 unit/μL, or 0.1 to about 1 unit/μL may be included in a reaction mixture.

Any patent, patent application, publication, or other disclosure material identified in the specification is hereby incorporated by reference herein in its entirety. Any material, or portion thereof, that is said to be incorporated by reference herein, but which conflicts with existing definitions, statements, or other disclosure material set forth herein is only incorporated to the extent that no conflict arises between that incorporated material and the present disclosure material.

### EXAMPLES

The following examples set forth methods for using the modified RNase H enzyme composition according to the present invention. It is understood that the steps of the methods described in these examples are not intended to be limiting. Further objectives and advantages of the present invention other than those set forth above will become apparent from the examples which are not intended to limit the scope of the present invention.

### Example 1

### Multiplex Assay of Salmonella invA Gene and E. Coli O157: H7 I Fragment

A multiplex assay of a Salmonella invA gene and an E. coli O157: H7 I fragment by real-time PCR was performed and an effect of the number of copies of target sequences on target sequence amplification was identified. Table 1 shows an example of a reaction mixture for a master mix used in real-time PCR:

TABLE 1
CataCleave Master Mix

| | | | | | | | | |
|---|---|---|---|---|---|---|---|---|
| #copies O157 I Plasmid: | 0 | 5.E+00 | 5.E+01 | 5.E+02 | 5.E+03 | 5.E+04 | 5.E+06 | 5.E+06 |
| Enter Number of Reactions: | 9 | 9 | 9 | 9 | 9 | 9 | 9 | 9 |
| Enter reaction volume (μL): | 25 | 25 | 25 | 25 | 25 | 25 | 25 | 25 |
| 10x I Buffer w/ 40 mM MgCl₂ | 22.5 | 22.5 | 22.5 | 22.5 | 22.5 | 22.5 | 22.5 | 22.5 |
| 2 mM dNTP mix (4 mM dUTP) | 9 | 9 | 9 | 9 | 9 | 9 | 9 | 9 |
| 25 μM inv-CC-Probe2(FAM) | 1.8 | 1.8 | 1.8 | 1.8 | 1.8 | 1.8 | 1.8 | 1.8 |
| 25 μM IAC-CC-Probe2(CYS) | 1.8 | 1.8 | 1.8 | 1.8 | 1.8 | 1.8 | 1.8 | 1.8 |
| 100 μM Salmonella-F1 Primer | 1.8 | 1.8 | 1.8 | 1.8 | 1.8 | 1.8 | 1.8 | 1.8 |
| 100 μM Sal-InvR2 Primer | 1.8 | 1.8 | 1.8 | 1.8 | 1.8 | 1.8 | 1.8 | 1.8 |
| 50 copies/μL Sal IAC2 Plasmid | 2.7 | 2.7 | 2.7 | 2.7 | 2.7 | 2.7 | 2.7 | 2.7 |
| 25 μM o057 I-P2 Probe (CY#) | 1.8 | 1.8 | 1.8 | 1.8 | 1.8 | 1.8 | 1.8 | 1.8 |
| 20 μM O157 I-F1 Primer | 9 | 9 | 9 | 9 | 9 | 9 | 9 | 9 |
| 20 μM O157 I-Primer | 9 | 9 | 9 | 9 | 9 | 9 | 9 | 9 |
| O157 I Plasmid | 0 | 9 | 9 | 9 | 9 | 9 | 9 | 9 |
| Ultrapure H₂O | 66.6 | 57.6 | 57.6 | 57.6 | 57.6 | 57.6 | 57.6 | 57.6 |
| Uracil DNA N-Glycosylase | 0.9 | 0.9 | 0.9 | 0.9 | 0.9 | 0.9 | 0.9 | 0.9 |
| RNase HII | 1.8 | 1.8 | 1.8 | 1.8 | 1.8 | 1.8 | 1.8 | 1.8 |
| Taq DNA Polymersase | 4.5 | 4.5 | 4.5 | 4.5 | 4.5 | 4.5 | 4.5 | 4.5 |
| Total Volume of Ready Mix | 135 | 135 | 135 | 135 | 135 | 135 | 135 | 135 |

The master mix shown in Table 1 was sufficient for eight reactions and for each reaction, 15 μl, of the reaction mixture was used. In Table 1, Sal IAC2 plasmid indicates a plasmid containing Salmonella internal amplification control, generated by inserting the internal amplification control probe (IAC-CC-Probe2) within a random sequence flanked by the Salmonella-specific primers (Salmonella-F1 and Sal-InvR2). E. coli O1571 plasmid indicates a plasmid containing E. coli O157: H7 I fragment, cloned from a non-pathogenic strain (ATCC 700728). Inv-CC-probe 2(FAM) (SEQ ID NO: 5), IAC-CC-probe2(CY5) (SEQ ID NO: 7), Salmonella-F1-primer (SEQ ID NO: 1), Sal-invR2 primer, (SEQ ID NO: 2), O157 I-P2 probe(CY3) (SEQ ID NO: 6), O157 I-F1 primer (SEQ ID NO: 3), and E. coli O157 I-R primer (SEQ ID NO: 4) were used in this example. The E. coli O157 I-P2 probe may be labeled with TYE563, which is equivalent to CY3. Uracil DNA N-glycosylase is an E. coli derived enzyme, overexpressed and purified. RNase HII is a Pyrococcus furiosus derived enzyme, overexpressed and purified from E. coli and a thermally stable enzyme. 10× I buffer is a HEPES-containing buffer (HEPES-KOH, MgCl₂, Kcl, BSA DMSO). Table 1 shows mixture compositions including different numbers of copies of the E. coli O157: H7 I plasmid. In the case of mixture compositions including different numbers of copies of the Sal invA plasmid, the same experiment was performed as described above, except that the number of copies of the E. coli O157: H7 I plasmid was fixed and the number of copies of the Sal invA plasmid varied. All plasmids were purified by CsCl gradient.

Conditions for real-time PCR were as follows: for UNG operation and denaturation, 10 minutes at a temperature of 37° C. and 10 minutes at a temperature of 95° C.; for denaturation, annealing and elongation, 15 seconds at a temperature of 95° C. and 20 seconds at a temperature of 60° C.; and for cooling, 10 seconds at a temperature of 4° C.

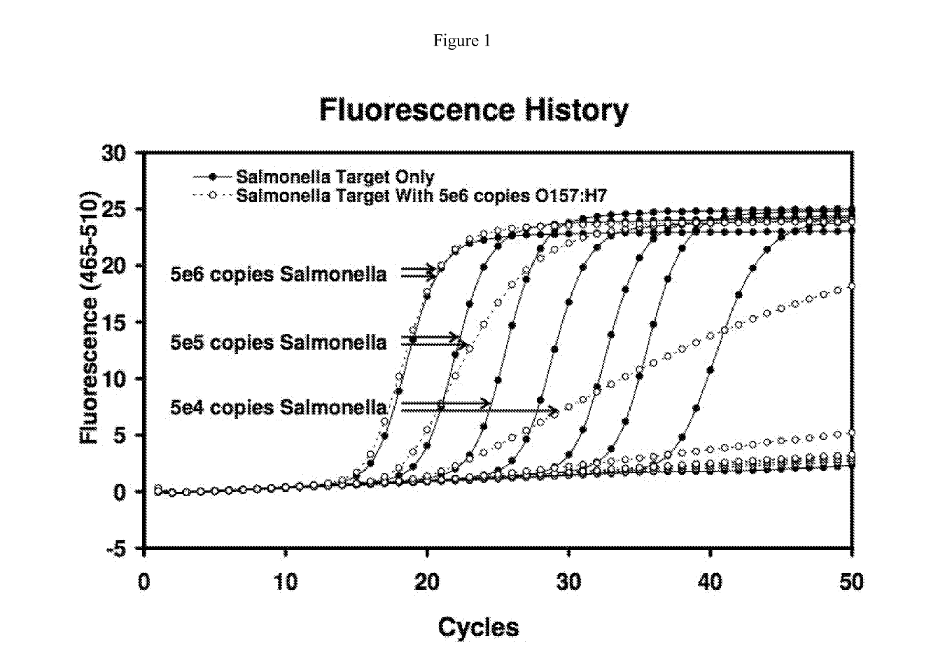

FIG. 1 shows real-time polymerase chain reaction (PCR) results when only a Salmonella target sequence exists and when a Salmonella target sequence and an E. coli O157: H7 target sequence coexist at different concentrations. Referring to FIG. 1, when only a Salmonella target sequence existed, a Cp value differed according to the number of copies of the Salmonella target sequence in a sample. However, when 5×10⁶ copies of the E. coli O157: H7 target sequence coexisted, the amplification of the Salmonella target sequence differed according to a ratio of the number of copies of the Salmonella target sequence to the number of copies of the E. coli O157: H7 target sequence. That is, when each of the Salmonella target sequence and the E. coli O157: H7 target sequence had 5×10⁶ copies, or when the Salmonella target sequence had 5×10⁵ copies and the E. coli O157: H7 target sequence had 5×10⁶ copies, that is, a ratio of the number of copies of the Salmonella target sequence to the number of copies of the E. coli O157: H7 target sequence was in the range of 1:1 to 1:10, the amplification was efficiently performed. However, when the number of copies of the Salmonella target sequence to the number of copies of the E. coli O157: H7 target sequence was in the range of 1:10 to 1: >10, the amplification was inefficiently performed.

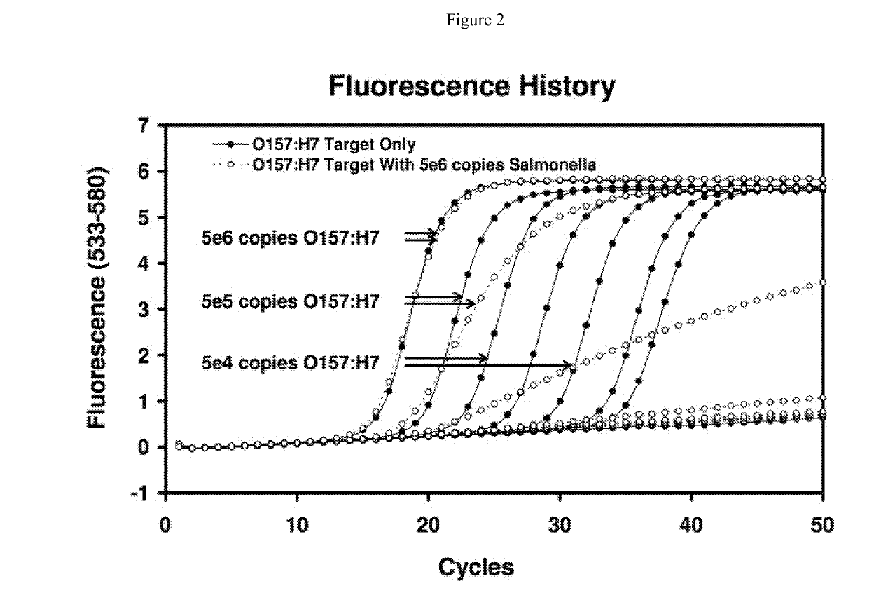

FIG. 2 shows real-time PCR results when only an E. coli O157: H7 target sequence exists and when a Salmonella target sequence and an E. coli O157: H7 target sequence coexist at different concentrations. Referring to FIG. 2, when only an E. coli O157: H7 target sequence existed, a Cp value differed according to the number of copies of the E. coli O157: H7 target sequence in a sample. However, when 5×10⁶ copies of the Salmonella target sequence coexisted, the amplification of the E. coli O157: H7 target sequence differed according to a ratio of the number of copies of the Salmonella target sequence to the number of copies of the E. coli O157: H7 target sequence. That is, when each of the E. coli O157: H7 target sequence and the Salmonella target sequence had 5×10⁶ copies, or when the E. coli O157: H7 target sequence had 5×10⁵ copies and the Salmonella target sequence had 5×10⁶ copies, that is, a ratio of the number of copies of the E. coli O157: H7 target sequence to the number of copies of the Salmonella target sequence was in the range of 1:1 to 1:10, the amplification was efficiently performed. However, when the number of copies of the E. coli O157: H7 target sequence to the number of copies of the Salmonella target sequence was in the range of 1:10 to 1: >10, the amplification was inefficiently performed.

Referring to FIGS. 1 and 2, it can be seen that when a plurality of target sequences are amplified together, the PCR amplification of one target sequence is dependent on the concentration of another target sequence.

In addition, a real-time PCR was performed using a 2.5 units/reaction of DNA Taq polymerase, 5, 5×10, 5×10³, 5×10⁴, 5×10⁵, and 5×10⁶ copies of Salmonella invA plasmid, and 5, 5×10, 5×10³, 5×10⁴, 5×10⁵, and 5×10⁶ copies of a E. coli O157: H7 I fragment.

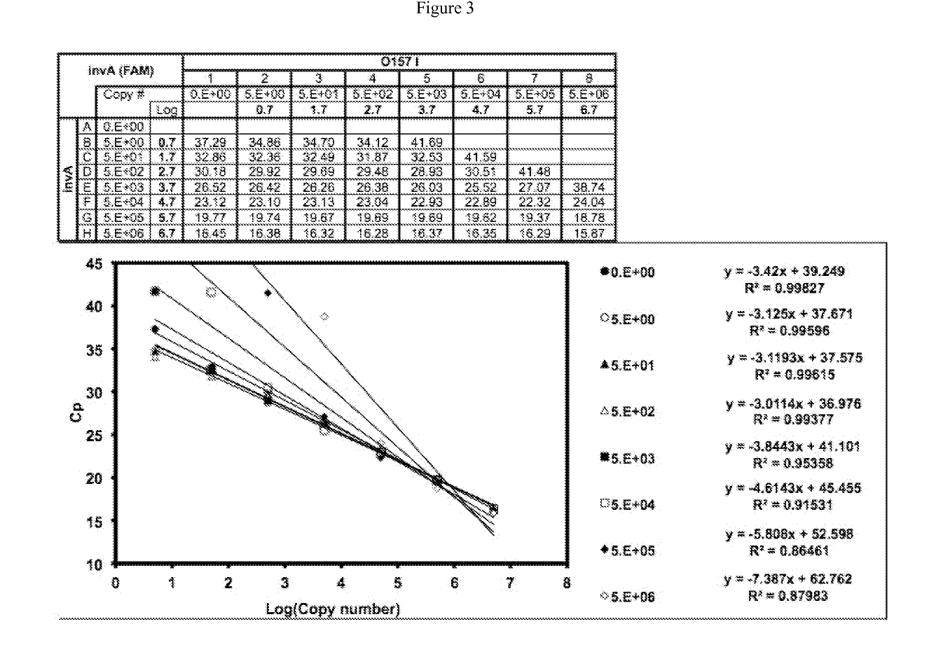

FIG. 3 is a graph of a Cp value with respect to the number of copies of the Salmonella invA plasmid target when the Salmonella invA plasmid and the E. coli O157: H7 I fragment exist at seven log concentrations and a low concentration of DNA Taq polymerase was used. Referring to FIG. 3, an x axis represents a log value with respect to the number of copies of the Salmonella invA plasmid, a y axis represents a Cp value, and 0.E+00 through 5.E+06 represent the number of copies of the E. coli O157: H7 I fragment.

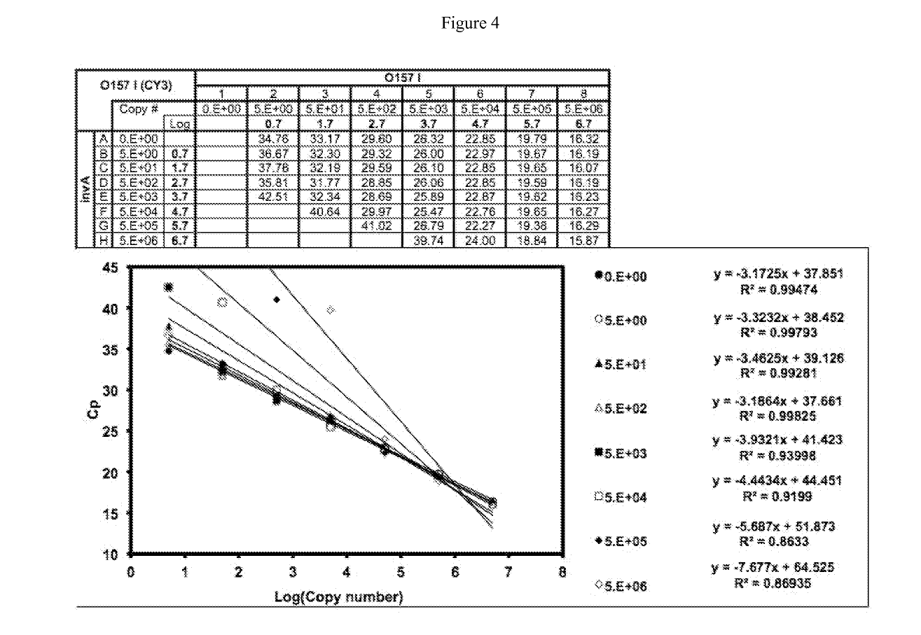

FIG. 4 is a graph of a Cp value with respect to the number of copies of the E. coli O157: H7 I fragment when the Salmonella invA plasmid and the E. coli O157: H7 I fragment exist at seven log concentrations and a low concentration of DNA Taq polymerase was used. Referring to FIG. 3, an x axis represents a log value with respect to the number of copies of the E. coli O157: H7 I fragment, a y axis represents a Cp value, and 0.E+00 through 5.E+06 represent the number of copies of the Salmonella invA plasmid.

Referring to FIGS. 3 and 4, when a low concentration of Taq DNA polymerase, for example, 2.5 units/reaction of Taq DNA polymerase, was used and the number of copies of one target sequence used was in the range of 5×10³ through 5×10⁶, the interrelationship of the number of copies of another target sequence with a Cp value was less significant.

### Example 2

### Effect of Concentration of DNA Polymerase on Multiplex Assay of Salmonella invA Gene and E. coli O157: H7 I Fragment

In this experiment, DNA polymerase having different concentrations was used to prevent one target sequence from affecting amplification of another target sequence when a plurality of target sequences are amplified by real-time PCR.

First, in order to prepare a high concentration of Taq polymerase, about 12 ml of Taq DNA polymerase (5 units/μL) was subjected to dialysis in a Taq storage buffer (50 mM Tris-HCl, pH 8.0, 100 mM KCl, 1 mM DTT, 0.1 mM EDTA) that did not include a surfactant, glycerol, or DTT. The resulting sample was concentrated to a volume of 450 μL by using a Microscon spin filter (Millipore). 100 μL of a 10×Taq storage buffer that did not include a surfactant or glycerol, and 450 μL glycerol were added to the concentrated sample to obtain a final volume of 1 ml.

Then, a real-time PCR was performed in the condition that the number of copies of Salmonella invA plasmid was 0 or 5×10⁶ and the number of copies of the E. coli O157: H7 I fragment plasmid varied. A PCR reaction mixture and condition were the same as in Example 1, except that 25 units of Taq DNA polymerase were used in a reaction with each reaction mixture.

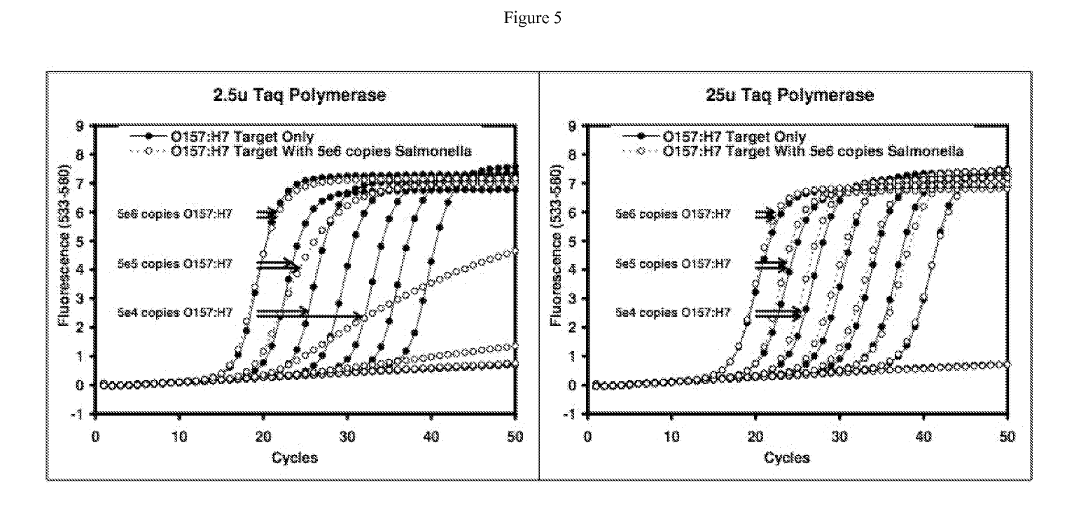

FIG. 5 is a graph showing an effect of the number of copies of the Salmonella invA plasmid on amplification of the E. coli O157: H7 I fragment, with respect to a DNA Taq polymerase. Referring to FIG. 5, in the case where 2.5 units/reaction of DNA Taq polymerase was used and 5×10⁶ copies of the Salmonella invA plasmid existed, when the number of copies of the E. coli O157: H7 I target was less than 5×10³, the E. coli O157: H7 I target was not detected (the left graph). On the other hand, in the case where 25 units/reaction of DNA Taq polymerase was used and 5×10⁶ copies of Salmonella invA plasmid existed, even when the number of copies of the E. coli O157: H7 I target is as small as 5, the E. coli O157: H7 I target was detected (the right graph).

In addition, a real-time PCR was performed using 25 units/reaction of DNA Taq polymerase, 5, 5×10, 5×10³, 5×10⁴, 5×10⁵, and 5×10⁶ copies of Salmonella invA plasmid, and 5, 5×10, 5×10³, 5×10⁴, 5×10⁵, and 5×10⁶ copies of E. coli O157: H7 I fragment.

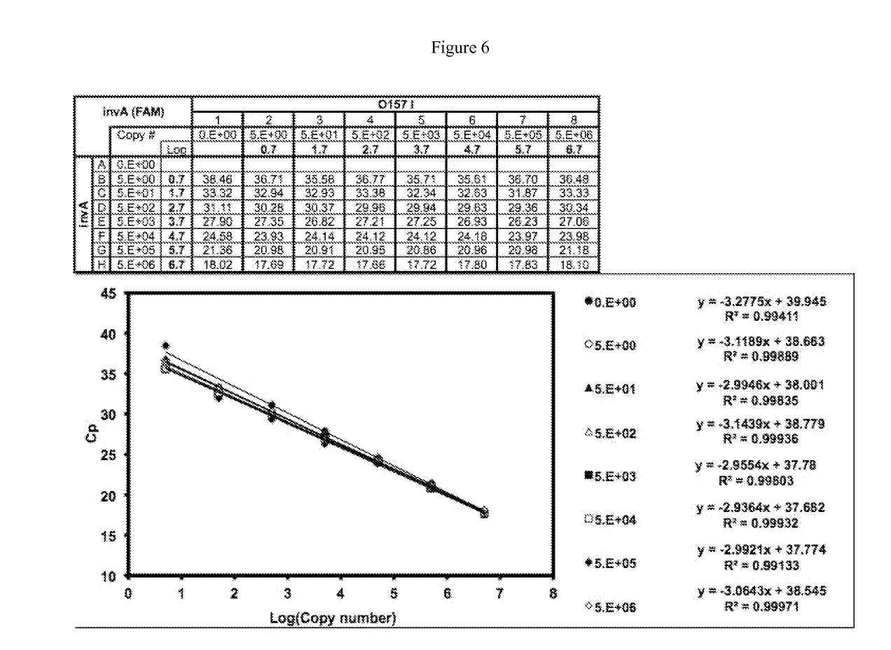

FIG. 6 is a graph of a Cp value with respect to the number of copies of the Salmonella invA plasmid target when the Salmonella invA plasmid and the E. coli O157: H7 I fragment exist at seven log concentrations and a high concentration of a DNA Taq polymerase was used. Referring to FIG. 6, an x axis represents a log value with respect to the number of copies of the Salmonella invA plasmid, a y axis represents a Cp value, and 0.E+00 through 5.E+06 represent the number of copies of the O157: H7 I fragment.

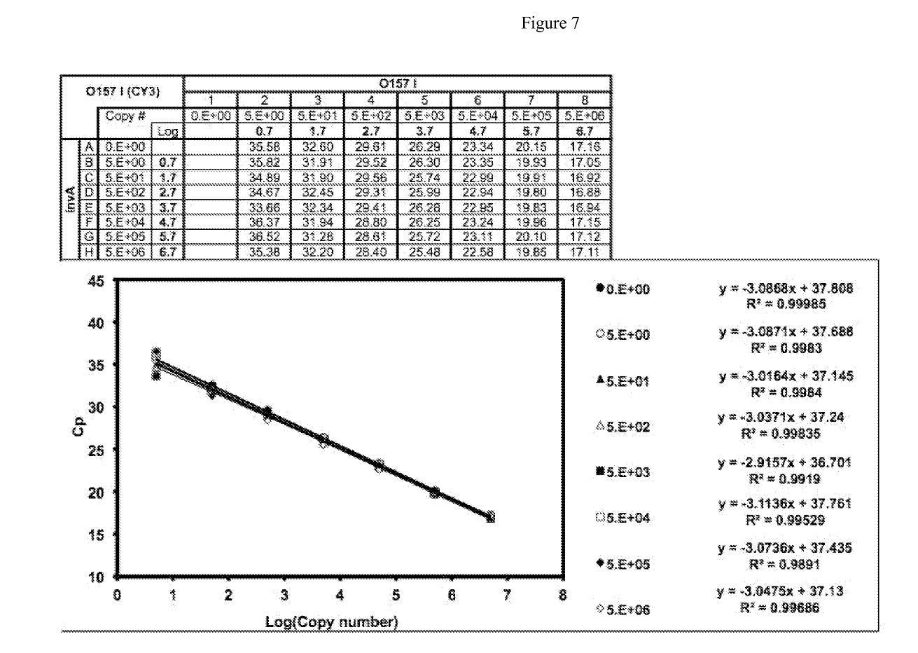

FIG. 7 is a graph of a Cp value with respect to the number of copies of the E. coli O157: H7 I fragment when the Salmonella invA plasmid and the E. coli O157: H7 I fragment exist at seven log concentrations and a high concentration of a DNA Taq polymerase was used. Referring to FIG. 7, an x axis represents a log value with respect to the number of copies of the E. coli O157: H7 I fragment, a y axis represents a Cp value, and 0.E+00 through 5.E+06 represent the number of copies of the Salmonella invA plasmid.

Referring to FIGS. 6 and 7, when a high concentration of Taq DNA polymerase, for example, 25 units/reaction of Taq DNA polymerase, was used, the interrelationship of the number of copies of another target sequence with a Cp value was significant when the number of copies of a target sequence was 5 to 5×10⁶.

According to a method of simultaneously amplifying target sequences from Salmonella spp. and E. coli O157: H7 in a sample, the target sequences of Salmonella spp. and E. coli O157: H7 in the sample may be simultaneously and efficiently amplified without interfering with each other. In addition, by using the method, Salmonella spp. and E. coli O157: H7 may also be detected in real time during the simultaneous and efficient amplification of the target sequences of Salmonella spp. and E. coli O157: H7 in the sample without interfering with each other.

While the present invention has been particularly shown and described with reference to exemplary embodiments thereof, it will be understood by those of ordinary skill in the art that various changes in form and details may be made therein without departing from the spirit and scope of the present invention as defined by the following claims.

1. A method of simultaneously detecting Salmonella spp. and E. coli O157: H7 in a sample comprising the steps of:
a) providing a sample to be tested for the presence of Salmonella spp. and E. coli O157: H7;
b) providing a pair of Salmonella-specific forward and reverse amplification primers that can anneal to a Salmonella-specific target DNA and a pair of E. coli O157: H7-specific amplification primers that can anneal to a E. coli O157: H7-specific target DNA;
c) amplifying a PCR fragment between the forward and reverse Salmonella-specific amplification primers and a PCR fragment between the first and second E. coli O157: H7-specific amplification primers in the presence of an amplifying polymerase activity and amplification buffer, wherein the concentration of the amplifying polymerase is equal to or higher than 0.1 unit/μl, and
d) detecting the Salmonella-specific and E. coli O157: H7-specific PCR amplification products,
wherein said detection of PCR amplification products indicates the presence of Salmonella and E. coli O157: H7 in said sample.

2. A method for the simultaneous, real-time PCR detection of Salmonella spp. and E. coli O157: H7 in a sample, comprising the steps of:
a) providing a sample to be tested for the presence of Salmonella and E. coli O157: H7;
b) providing a pair of Salmonella-specific forward and reverse amplification primers that can anneal to a Salmonella-specific target DNA and a pair of E. coli O157: H7-specific forward and reverse amplification primers that can anneal to a E. coli O157: H7-specific target DNA;
c) providing a Salmonella-specific probe and an E. coli O157: H7-specific probe, each probe comprising a detectable label and DNA and RNA nucleic acid sequences that are substantially complimentary to either the Salmonella-specific or E. coli O157: H7-specific target DNAs respectively;
d) amplifying a PCR fragment between the Salmonella-specific forward and reverse amplification primers and amplifying a PCR fragment between the E. coli O157: H7-specific forward and reverse amplification primers in the presence of an amplifying polymerase activity, amplification buffer; an RNAse H activity and the Salmonella-specific and E. coli O157: H7-specific probes under conditions where the RNA sequences within each probe can form a RNA: DNA heteroduplex with a complimentary target DNA sequences in the PCR fragments, and
e) detecting a real-time increase in the emission of a signal from the label on the Salmonella-specific and E. coli O157: H7-specific probes,
wherein the increase in signal indicates the presence of Salmonella and E. coli O157: H7 in the sample.

3. The method of claim 2, wherein the real-time increase in the emission of the signal from the label on the Salmonella-specific and E. coli O157: H7-specific probes results from:
the RNAse H cleavage of the RNA: DNA heteroduplex formed between the RNA sequences of the Salmonella-specific probes and one of the strands of the Salmonella-specific PCR fragments and
the RNAse H cleavage of the RNA: DNA heteroduplex formed between the RNA sequences of the E. coli O157: H7-specific probes and one of the strands of the E. coli O157: H7-specific PCR fragments.

4. The method of claim 2, wherein the DNA and RNA sequences of the Salmonella-specific and the E. coli O157: H7-specific probes are covalently linked.

5. The method of claim 2, wherein the Salmonella-specific and E. coli O157: H7-specific probes are labeled with a FRET pair.

6. The method of claim 2, wherein the sample comprises a food sample.

7. The method of claim 2, wherein the sample comprises a surface wipe sample.

8. The method of claim 2, wherein the nucleic acid within the sample is pre-treated with uracil-N-glycosylase.

9. The method of claim 8, wherein the uracil-N-glycosylase is inactivated prior to PCR amplification.

10. The method of claim 2, wherein the Salmonella-specific probe has a structure of R1-X-R2 and the E. coli O157: H7-specific probe has a structure of R1′-X-R2′, wherein R1, R1′, R2 and R2′ are each selected from the group consisting of a nucleic acid and a nucleic acid analog, and X is a first RNA, and the R1, R1′, R2 and R2′ each are coupled to a detectable label.

11. The method of claim 2, wherein the pair of Salmonella-specific amplification primers comprises a forward primer (SEQ ID NO: 1) and a reverse primer ((SEQ ID NO: 2), and the pair of E. coli O157: H7-specific amplification primers comprises a forward primer (SEQ ID NO: 3) and a reverse primer ((SEQ ID NO: 4).

12. The method of claim 2, wherein the Salmonella-specific probe has a nucleotide sequence of SEQ ID NO: 5 and the E. coli O157: H7-specific probe has a nucleotide sequence of SEQ ID NO: 6.

13. A kit for simultaneously amplifying and detecting target sequences from Salmonella and E. coli O157: H7 in a sample comprising:
a pair of Salmonella-specific forward and reverse amplification primers,
a pair of E. coli O157: H7-specific forward and reverse amplification primers,
a Salmonella-specific probe which has a structure of R1-X-R2,
an E. coli O157: H7-specific probe which has a structure of R1′-X-R2′,
a RNase H, and
an amplifying polymerase activity,
wherein R1, R1′, R2 and R2′ are each selected from the group consisting of a nucleic acid and a nucleic acid analog, and X may be a first RNA, and the R1, R1′, R2 and R2′ each are coupled to a detectable label.

14. The kit of claim 13, wherein the pair of Salmonella-specific amplification forward and reverse primers comprises a forward primer (SEQ ID NO: 1) and a reverse primer ((SEQ ID NO: 2), and the pair of E. coli O157: H7-specific amplification primers comprises a forward primer (SEQ ID NO: 3) and a reverse primer ((SEQ ID NO: 4).

15. The kit of claim 13, wherein the Salmonella-specific probe has a nucleotide sequence of SEQ ID NO: 5 and the E. coli O157: H7-specific probe has a nucleotide sequence of SEQ ID NO: 6.

16. The kit of claim 13, wherein the DNA and RNA sequences of the Salmonella-specific and the E. coli O157: H7-specific probes are covalently linked.

17. The kit of claim 13, wherein the Salmonella-specific and E. coli O157: H7-specific probes are labeled with a fluorescent label.

18. The kit of claim 13, wherein the Salmonella-specific and E. coli O157: H7-specific probes each are labeled with a FRET pair.

19. The kit of claim 13, wherein the Salmonella-specific and E. coli O157: H7-specific probes are linked to a solid support.

20. The kit of claim 13, further comprising uracil-N-glycosylase.
